(19) United States
(12) Patent Application Publication
(10) Pub. No.: US 2024/0395411 A1
(43) Pub. Date: Nov. 28, 2024

(54) FORWARD LOOKING HEALTH-RELATED PREDICTION

(71) Applicant: Oura Health Oy, Oulu (FI)

(72) Inventors: Laura Furman, San Francisco, CA (US); Jillian Ann Peacock, Oakland, CA (US); Shishir Bhattarai, Helsinki (FI); Jake Harris Sherman, Seattle, WA (US); Andrew Paul Fischer, Denver, CO (US); Colleen Diane Simms, Scottsdale, AZ (US)

(21) Appl. No.: 18/322,941

(22) Filed: May 24, 2023

(51) Int. Cl. G16H 50/30; G16H 40/67

(52) U.S. Cl. CPC G16H 50/30; G16H 40/67

(57) ABSTRACT

Methods, systems, and devices for generating personalized health-related predictions from measured physiological data are described. A system may receive, from a wearable device, first physiological data measured from a user via the wearable device through the first time interval. The system may output, via a machine learning model and based on the first physiological data, one or more health related predictions associated with the user during a second time interval. The one or more health-related predictions may include a predicted change in a health related metric during the second time interval based on one or more hypothetical user actions (e.g., expected or anticipated user actions) engaged in by the user between the first time interval and a second time interval. As such, a user interface of a user device associated with the wearable device may display information associated with the one or more health-related predictions prior to the second time interval.

### FIELD OF TECHNOLOGY

The following relates to wearable devices and data processing, including forward looking health-related predictions.

### BACKGROUND

Some wearable devices may be configured to collect physiological data from users, and display information back to the user regarding their physiological data. In this regard, information provided by many wearable devices is primarily backwards-looking, in that information displayed to the user is based on actions that the user performed in the past, and data collected by the wearable device in the past. However, such backwards-looking data is not compelling to some users, leading to a lack of engagement between the users and the wearable device.

### BRIEF DESCRIPTION OF THE DRAWINGS

FIG. 1 illustrates an example of a system that supports forward looking health-related prediction in accordance with aspects of the present disclosure.

FIG. 2 illustrates an example of a system that supports forward looking health-related prediction in accordance with aspects of the present disclosure.

FIG. 3 shows an example of a timeline that supports forward looking health-related prediction in accordance with aspects of the present disclosure.

FIG. 4 shows an example of a graphical user interface (GUI) that supports forward looking health-related prediction in accordance with aspects of the present disclosure.

FIG. 5 shows a block diagram of an apparatus that supports forward looking health-related prediction in accordance with aspects of the present disclosure.

FIG. 6 shows a block diagram of a wearable application that supports forward looking health-related prediction in accordance with aspects of the present disclosure.

FIG. 7 shows a diagram of a system including a user device that supports forward looking health-related prediction in accordance with aspects of the present disclosure.

FIG. 8 shows a diagram of a system including a wearable device that supports forward looking health-related prediction in accordance with aspects of the present disclosure.

FIGS. 9 and 10 show flowcharts illustrating methods that support forward looking health-related prediction in accordance with aspects of the present disclosure.

### DETAILED DESCRIPTION

Wearable devices may be configured to collect physiological data from users, and display information back to the user regarding their physiological data. In this regard, information provided by many wearable devices is primarily backwards-looking, in that information displayed to the user is based on actions that the user performed in the past, and data collected by the wearable device in the past. However, such backwards-looking data is not compelling to some users, leading to a lack of engagement between the users and the wearable device. That is, for some users, viewing physiological data from the past may not provide enough motivation for the users to proactively make decisions to improve their health in the future. To address this issue, some wearable devices may provide forward-looking recommendations to help the user improve their overall health going forward, such as suggestions for the user to adjust their bedtime this evening, or to take a walk after dinner to improve their sleep quality for the upcoming night. However, such forward-looking recommendations are largely speculative, and are insufficient to motivate users to take proactive steps to improve their health. In other words, users are generally aware that going to bed earlier and getting more exercise will improve their sleep quality, but may still be unmotivated to make these lifestyle choices due to the fact that the benefits provided from such choices are somewhat nebulous and hard to define.

Accordingly, techniques described herein may enable generation of forward-looking health-related predictions. In particular, a system may utilize historical physiological data collected from a user via a wearable device to make predictions regarding the user's physiological data in the future. For example, the system may take the user's physiological data from the current day (e.g., activity data, heart rate variability (HRV) data, heart rate data, etc.), and predict what the user's Sleep Score will be for the next day. In some cases, the system may utilize a machine learning model to make such predictions, and may leverage data collected from other, similar users to improve predictions. Such forward-looking predictions may provide some users with more motivation to proactively make choices to improve their overall health.

In some cases, the system may provide suggestions for hypothetical (e.g., expected, predicted, anticipated) user actions and/or hypothetical feelings or emotional states of being (e.g., relaxation, stress, anxiety) in the future, and may indicate how the user's future physiological data will be affected by following such hypothetical suggestions. For example, the system may recommend that a user take a walk after dinner, and may indicate that the walk is expected to improve the user's Sleep Score for the upcoming night by three points. Similarly, in some implementations, a user may be able to hypothetically “adjust” or input their hypothetical future actions (e.g., expected or anticipated user actions), and the system may indicate how such future actions may affect the user's future physiological data. For example, a graphical user interface (GUI) may display a slide bar representing the user's bedtime for the upcoming night, where the user is able to move the slide bar to indicate different hypothetical/expected bedtimes. In this example, as the user moves the slide bar to different hypothetical/expected bedtimes, the system may indicate how the user's Sleep Score for the next day is expected to change based on the indicated bedtimes (e.g., “If you go to sleep at 10:00 pm, we expect your Sleep Score to be 89. If you go to sleep at 9:00 pm, we expect your Sleep Score to be 93.”

For the purposes of the present disclosure, the terms “hypothetical user actions,” “expected/predicted user actions,” “hypothetical emotions/states,” “expected/predicted emotions/states,” and like terms, may refer to actions and states/emotions (e.g., relaxation, stress) that are expected or predicted to occur at some point in the future. In particular, systems described herein may be able to predict or anticipate future user actions or emotions/states of being, and use the predicted user actions and/or predicted emotions/states of being to make health-related predictions for the user.

Aspects of the disclosure are initially described in the context of systems supporting physiological data collection from users via wearable devices. Aspects of the disclosure are then described in the context of a GUI. Aspects of the disclosure are further illustrated by and described with reference to apparatus diagrams, system diagrams, and flowcharts that relate to forward looking health-related prediction.

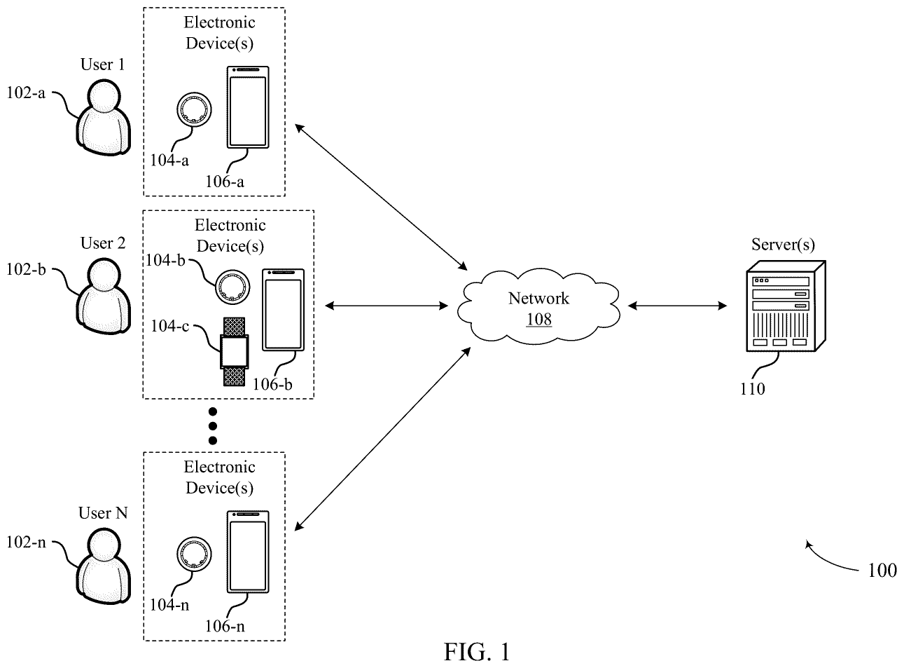

FIG. 1 illustrates an example of a system 100 that supports forward looking health-related prediction in accordance with aspects of the present disclosure. The system 100 includes a plurality of electronic devices (e.g., wearable devices 104, user devices 106) that may be worn and/or operated by one or more users 102. The system 100 further includes a network 108 and one or more servers 110.

The electronic devices may include any electronic devices known in the art, including wearable devices 104 (e.g., ring wearable devices, watch wearable devices, etc.), user devices 106 (e.g., smartphones, laptops, tablets). The electronic devices associated with the respective users 102 may include one or more of the following functionalities: 1) measuring physiological data, 2) storing the measured data, 3) processing the data, 4) providing outputs (e.g., via GUIs) to a user 102 based on the processed data, and 5) communicating data with one another and/or other computing devices. Different electronic devices may perform one or more of the functionalities.

Example wearable devices 104 may include wearable computing devices, such as a ring computing device (hereinafter “ring”) configured to be worn on a user's 102 finger, a wrist computing device (e.g., a smart watch, fitness band, or bracelet) configured to be worn on a user's 102 wrist, and/or a head mounted computing device (e.g., glasses/goggles). Wearable devices 104 may also include bands, straps (e.g., flexible or inflexible bands or straps), stick-on sensors, and the like, that may be positioned in other locations, such as bands around the head (e.g., a forehead headband), arm (e.g., a forearm band and/or bicep band), and/or leg (e.g., a thigh or calf band), behind the ear, under the armpit, and the like. Wearable devices 104 may also be attached to, or included in, articles of clothing. For example, wearable devices 104 may be included in pockets and/or pouches on clothing. As another example, wearable device 104 may be clipped and/or pinned to clothing, or may otherwise be maintained within the vicinity of the user 102. Example articles of clothing may include, but are not limited to, hats, shirts, gloves, pants, socks, outerwear (e.g., jackets), and undergarments. In some implementations, wearable devices 104 may be included with other types of devices such as training/sporting devices that are used during physical activity. For example, wearable devices 104 may be attached to, or included in, a bicycle, skis, a tennis racket, a golf club, and/or training weights.

Much of the present disclosure may be described in the context of a ring wearable device 104. Accordingly, the terms “ring 104,” “wearable device 104,” and like terms, may be used interchangeably, unless noted otherwise herein. However, the use of the term “ring 104” is not to be regarded as limiting, as it is contemplated herein that aspects of the present disclosure may be performed using other wearable devices (e.g., watch wearable devices, necklace wearable device, bracelet wearable devices, earring wearable devices, anklet wearable devices, and the like).

In some aspects, user devices 106 may include handheld mobile computing devices, such as smartphones and tablet computing devices. User devices 106 may also include personal computers, such as laptop and desktop computing devices. Other example user devices 106 may include server computing devices that may communicate with other electronic devices (e.g., via the Internet). In some implementations, computing devices may include medical devices, such as external wearable computing devices (e.g., Holter monitors). Medical devices may also include implantable medical devices, such as pacemakers and cardioverter defibrillators. Other example user devices 106 may include home computing devices, such as internet of things (IoT) devices (e.g., IoT devices), smart televisions, smart speakers, smart displays (e.g., video call displays), hubs (e.g., wireless communication hubs), security systems, smart appliances (e.g., thermostats and refrigerators), and fitness equipment.

Some electronic devices (e.g., wearable devices 104, user devices 106) may measure physiological parameters of respective users 102, such as photoplethysmography waveforms, continuous skin temperature, a pulse waveform, respiration rate, heart rate, heart rate variability (HRV), actigraphy, galvanic skin response, pulse oximetry, blood oxygen saturation (SpO2), blood sugar levels (e.g., glucose metrics), and/or other physiological parameters. Some electronic devices that measure physiological parameters may also perform some/all of the calculations described herein. Some electronic devices may not measure physiological parameters, but may perform some/all of the calculations described herein. For example, a ring (e.g., wearable device 104), mobile device application, or a server computing device may process received physiological data that was measured by other devices.

In some implementations, a user 102 may operate, or may be associated with, multiple electronic devices, some of which may measure physiological parameters and some of which may process the measured physiological parameters. In some implementations, a user 102 may have a ring (e.g., wearable device 104) that measures physiological parameters. The user 102 may also have, or be associated with, a user device 106 (e.g., mobile device, smartphone), where the wearable device 104 and the user device 106 are communicatively coupled to one another. In some cases, the user device 106 may receive data from the wearable device 104 and perform some/all of the calculations described herein. In some implementations, the user device 106 may also measure physiological parameters described herein, such as motion/activity parameters.

For example, as illustrated in FIG. 1, a first user 102-a (User 1) may operate, or may be associated with, a wearable device 104-a (e.g., ring 104-a) and a user device 106-a that may operate as described herein. In this example, the user device 106-a associated with user 102-a may process/store physiological parameters measured by the ring 104-a. Comparatively, a second user 102-b (User 2) may be associated with a ring 104-b, a watch wearable device 104-c (e.g., watch 104-c), and a user device 106-b, where the user device 106-b associated with user 102-b may process/store physiological parameters measured by the ring 104-b and/or the watch 104-c. Moreover, an nth user 102-n (User N) may be associated with an arrangement of electronic devices described herein (e.g., ring 104-n, user device 106-n). In some aspects, wearable devices 104 (e.g., rings 104, watches 104) and other electronic devices may be communicatively coupled to the user devices 106 of the respective users 102 via Bluetooth, Wi-Fi, and other wireless protocols.

In some implementations, the rings 104 (e.g., wearable devices 104) of the system 100 may be configured to collect physiological data from the respective users 102 based on arterial blood flow within the user's finger. In particular, a ring 104 may utilize one or more light-emitting components, such as LEDs (e.g., red LEDs, green LEDs) that emit light on the palm-side of a user's finger to collect physiological data based on arterial blood flow within the user's finger. In general, the terms light-emitting components, light-emitting elements, and like terms, may include, but are not limited to, LEDs, micro LEDs, mini LEDs, laser diodes (LDs) (e.g., vertical cavity surface-emitting lasers (VCSELs), and the like.

In some cases, the system 100 may be configured to collect physiological data from the respective users 102 based on blood flow diffused into a microvascular bed of skin with capillaries and arterioles. For example, the system 100 may collect PPG data based on a measured amount of blood diffused into the microvascular system of capillaries and arterioles. In some implementations, the ring 104 may acquire the physiological data using a combination of both green and red LEDs. The physiological data may include any physiological data known in the art including, but not limited to, temperature data, accelerometer data (e.g., movement/motion data), heart rate data, HRV data, blood oxygen level data, or any combination thereof.

The use of both green and red LEDs may provide several advantages over other solutions, as red and green LEDs have been found to have their own distinct advantages when acquiring physiological data under different conditions (e.g., light/dark, active/inactive) and via different parts of the body, and the like. For example, green LEDs have been found to exhibit better performance during exercise. Moreover, using multiple LEDs (e.g., green and red LEDs) distributed around the ring 104 has been found to exhibit superior performance as compared to wearable devices that utilize LEDs that are positioned close to one another, such as within a watch wearable device. Furthermore, the blood vessels in the finger (e.g., arteries, capillaries) are more accessible via LEDs as compared to blood vessels in the wrist. In particular, arteries in the wrist are positioned on the bottom of the wrist (e.g., palm-side of the wrist), meaning only capillaries are accessible on the top of the wrist (e.g., back of hand side of the wrist), where wearable watch devices and similar devices are typically worn. As such, utilizing LEDs and other sensors within a ring 104 has been found to exhibit superior performance as compared to wearable devices worn on the wrist, as the ring 104 may have greater access to arteries (as compared to capillaries), thereby resulting in stronger signals and more valuable physiological data.

The electronic devices of the system 100 (e.g., user devices 106, wearable devices 104) may be communicatively coupled to one or more servers 110 via wired or wireless communication protocols. For example, as shown in FIG. 1, the electronic devices (e.g., user devices 106) may be communicatively coupled to one or more servers 110 via a network 108. The network 108 may implement transfer control protocol and internet protocol (TCP/IP), such as the Internet, or may implement other network 108 protocols. Network connections between the network 108 and the respective electronic devices may facilitate transport of data via email, web, text messages, mail, or any other appropriate form of interaction within a computer network 108. For example, in some implementations, the ring 104-a associated with the first user 102-a may be communicatively coupled to the user device 106-a, where the user device 106-a is communicatively coupled to the servers 110 via the network 108. In additional or alternative cases, wearable devices 104 (e.g., rings 104, watches 104) may be directly communicatively coupled to the network 108.

The system 100 may offer an on-demand database service between the user devices 106 and the one or more servers 110. In some cases, the servers 110 may receive data from the user devices 106 via the network 108, and may store and analyze the data. Similarly, the servers 110 may provide data to the user devices 106 via the network 108. In some cases, the servers 110 may be located at one or more data centers. The servers 110 may be used for data storage, management, and processing. In some implementations, the servers 110 may provide a web-based interface to the user device 106 via web browsers.

In some aspects, the system 100 may detect periods of time that a user 102 is asleep, and classify periods of time that the user 102 is asleep into one or more sleep stages (e.g., sleep stage classification). For example, as shown in FIG. 1, User 102-a may be associated with a wearable device 104-a (e.g., ring 104-a) and a user device 106-a. In this example, the ring 104-a may collect physiological data associated with the user 102-a, including temperature, heart rate, HRV, respiratory rate, and the like. In some aspects, data collected by the ring 104-a may be input to a machine learning classifier, where the machine learning classifier is configured to determine periods of time that the user 102-a is (or was) asleep. Moreover, the machine learning classifier may be configured to classify periods of time into different sleep stages, including an awake sleep stage, a rapid eye movement (REM) sleep stage, a light sleep stage (non-REM (NREM)), and a deep sleep stage (NREM). In some aspects, the classified sleep stages may be displayed to the user 102-a via a GUI of the user device 106-a. Sleep stage classification may be used to provide feedback to a user 102-a regarding the user's sleeping patterns, such as recommended bedtimes, recommended wake-up times, and the like. Moreover, in some implementations, sleep stage classification techniques described herein may be used to calculate scores for the respective user, such as Sleep Scores, Readiness Scores, and the like.

In some aspects, the system 100 may utilize circadian rhythm-derived features to further improve physiological data collection, data processing procedures, and other techniques described herein. The term circadian rhythm may refer to a natural, internal process that regulates an individual's sleep-wake cycle, that repeats approximately every 24 hours. In this regard, techniques described herein may utilize circadian rhythm adjustment models to improve physiological data collection, analysis, and data processing. For example, a circadian rhythm adjustment model may be input into a machine learning classifier along with physiological data collected from the user 102-a via the wearable device 104-a. In this example, the circadian rhythm adjustment model may be configured to “weight.” or adjust, physiological data collected throughout a user's natural, approximately 24-hour circadian rhythm. In some implementations, the system may initially start with a “baseline” circadian rhythm adjustment model, and may modify the baseline model using physiological data collected from each user 102 to generate tailored, individualized circadian rhythm adjustment models that are specific to each respective user 102.

In some aspects, the system 100 may utilize other biological rhythms to further improve physiological data collection, analysis, and processing by phase of these other rhythms. For example, if a weekly rhythm is detected within an individual's baseline data, then the model may be configured to adjust “weights” of data by day of the week. Biological rhythms that may require adjustment to the model by this method include: 1) ultradian (faster than a day rhythms, including sleep cycles in a sleep state, and oscillations from less than an hour to several hours periodicity in the measured physiological variables during wake state; 2) circadian rhythms; 3) non-endogenous daily rhythms shown to be imposed on top of circadian rhythms, as in work schedules; 4) weekly rhythms, or other artificial time periodicities exogenously imposed (e.g., in a hypothetical culture with 12 day “weeks,” 12 day rhythms could be used); 5) multi-day ovarian rhythms in women and spermatogenesis rhythms in men; 6) lunar rhythms (relevant for individuals living with low or no artificial lights); and 7) seasonal rhythms.

The biological rhythms are not always stationary rhythms. For example, many women experience variability in ovarian cycle length across cycles, and ultradian rhythms are not expected to occur at exactly the same time or periodicity across days even within a user. As such, signal processing techniques sufficient to quantify the frequency composition while preserving temporal resolution of these rhythms in physiological data may be used to improve detection of these rhythms, to assign phase of each rhythm to each moment in time measured, and to thereby modify adjustment models and comparisons of time intervals. The biological rhythm-adjustment models and parameters can be added in linear or non-linear combinations as appropriate to more accurately capture the dynamic physiological baselines of an individual or group of individuals.

In some aspects, the respective devices of the system 100 may support techniques for generation of forward-looking health-related predictions. In particular, the system 100 may utilize historical physiological data collected from a user, such as the user 102-a, the user 102-b, or the user 102-c, via a wearable device 104 associated with the respective user 102, to make predictions regarding the physiological data associated with the user 102 in the future.

For example, the system 100 may take the user's physiological data from the current day (e.g., activity data, HRV data, heart rate data, etc.), and predict what the user's Sleep Score will be for the next day. In some cases, the system 100 may utilize a machine learning model (e.g., run on a server 110) to make such predictions, and may leverage data collected from other, similar users 102 to improve predictions.

For example, the system 100 may collect first physiological data associated with the user 102-b via the ring 104-b, the watch 104-c, or both, during a first time interval, such as a previous night, to generate a first value of a health-related metric associated with the user 102-b, such as a Sleep Score of the user 102-b. In other words, the system 100 may predict what the user's Sleep Score will be for the following day based on the user's previous physiological data collected during the previous 24 hours (or some other time period). Additionally, the system 100 may predict how the Sleep Score for the following day is expected to change based on hypothetical (e.g., expected, predicted) user actions that the user may engage in at some time in the future, and/or expected emotions/states of being that the user is expected to experience at some point in the future.

For example, the system 100 may predict that the user's Sleep Score for the following day will increase by 2 points if the user goes to bed tonight one hour earlier than their normal bedtime. In this regard, the system 100 may take the user's previously-collected physiological data to make predictions as to how the user's physiological data (e.g., Sleep Score, Readiness Score, HRV, heart rate) is expected to change at some time in the future, such as the following day. By way of another example, the system 100 may recognize that the user has a therapy session scheduled for later that day (such as based on a calendar application executable via the user device 106), and that the user is therefore expected to experience a range of emotions and states of being during the therapy session (e.g., relief, anger, stress, anxiety, etc.). In this example, the system 100 may take the expected emotions/states of being into account when predicting the user's sleep score (and/or other health-related parameters) for the user at some point in the future.

The machine learning model may generate the prediction based on one or more relationships between the collected data and the health-related metric. For example, the system 100 may identify that caffeine consumed after 5 pm typically results in a reduction in sleep quality, thus reducing a Sleep Score of the user 102. In some examples, the one or more relationships may be based on historical data (e.g., trends) associated with the user 102-b. For example, the system 100 may identify the relationship between caffeine consumption and sleep quality for the user 102-b based on previous instances of the user 102-b consuming caffeine after 5 pm. Additionally, or alternatively, the one or more relationships may be based on historical data associated with a set of users 102. For example, the system may identify that both the user 102-a and the user 102-c experienced a reduction in sleep quality based on caffeine consumption after 5 pm. As such, the prediction of the second value of the health-related metric for the user 102-b may be based on the one or more relationships identified based on the user 102-a and the user 102-c. Such forward-looking predictions may provide some users with more motivation to proactively make choices to improve their overall health.

It should be appreciated by a person skilled in the art that one or more aspects of the disclosure may be implemented in a system 100 to additionally or alternatively solve other problems than those described above. Furthermore, aspects of the disclosure may provide technical improvements to “conventional” systems or processes as described herein. However, the description and appended drawings only include example technical improvements resulting from implementing aspects of the disclosure, and accordingly do not represent all of the technical improvements provided within the scope of the claims.

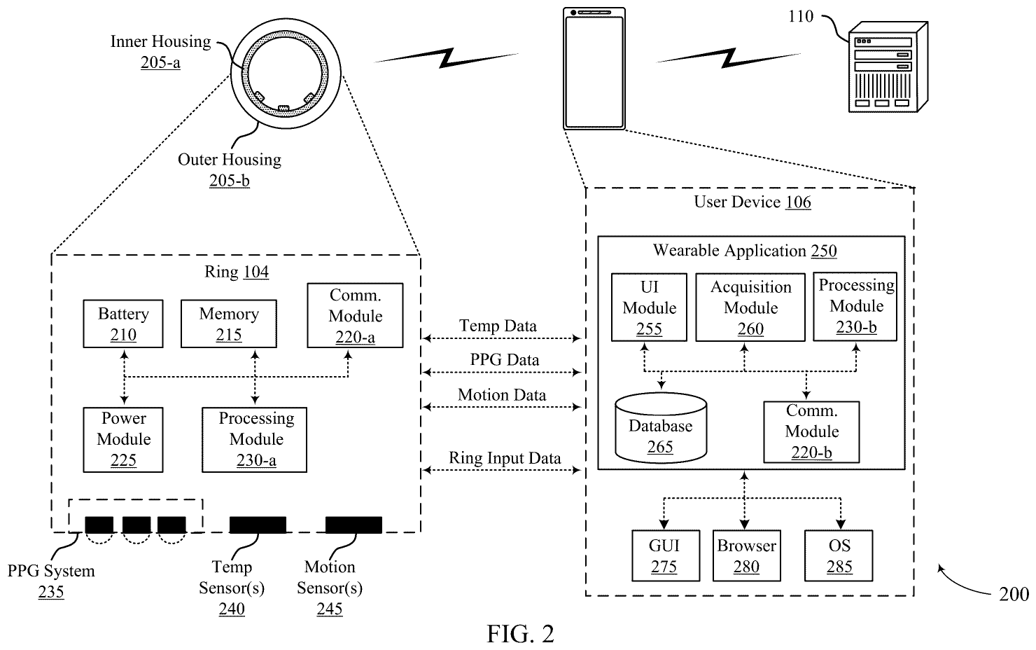

FIG. 2 illustrates an example of a system 200 that supports forward looking health-related prediction in accordance with aspects of the present disclosure. The system 200 may implement, or be implemented by, system 100. In particular, system 200 illustrates an example of a ring 104 (e.g., wearable device 104), a user device 106, and a server 110, as described with reference to FIG. 1.

In some aspects, the ring 104 may be configured to be worn around a user's finger, and may determine one or more user physiological parameters when worn around the user's finger. Example measurements and determinations may include, but are not limited to, user skin temperature, pulse waveforms, respiratory rate, heart rate, HRV, blood oxygen levels (SpO2), blood sugar levels (e.g., glucose metrics), and the like.

The system 200 further includes a user device 106 (e.g., a smartphone) in communication with the ring 104. For example, the ring 104 may be in wireless and/or wired communication with the user device 106. In some implementations, the ring 104 may send measured and processed data (e.g., temperature data, photoplethysmogram (PPG) data, motion/accelerometer data, ring input data, and the like) to the user device 106. The user device 106 may also send data to the ring 104, such as ring 104 firmware/configuration updates. The user device 106 may process data. In some implementations, the user device 106 may transmit data to the server 110 for processing and/or storage.

The ring 104 may include a housing 205 that may include an inner housing 205-a and an outer housing 205-b. In some aspects, the housing 205 of the ring 104 may store or otherwise include various components of the ring including, but not limited to, device electronics, a power source (e.g., battery 210, and/or capacitor), one or more substrates (e.g., printable circuit boards) that interconnect the device electronics and/or power source, and the like. The device electronics may include device modules (e.g., hardware/software), such as: a processing module 230-a, a memory 215, a communication module 220-a, a power module 225, and the like. The device electronics may also include one or more sensors. Example sensors may include one or more temperature sensors 240, a PPG sensor assembly (e.g., PPG system 235), and one or more motion sensors 245.

The sensors may include associated modules (not illustrated) configured to communicate with the respective components/modules of the ring 104, and generate signals associated with the respective sensors. In some aspects, each of the components/modules of the ring 104 may be communicatively coupled to one another via wired or wireless connections. Moreover, the ring 104 may include additional and/or alternative sensors or other components that are configured to collect physiological data from the user, including light sensors (e.g., LEDs), oximeters, and the like.

The ring 104 shown and described with reference to FIG. 2 is provided solely for illustrative purposes. As such, the ring 104 may include additional or alternative components as those illustrated in FIG. 2. Other rings 104 that provide functionality described herein may be fabricated. For example, rings 104 with fewer components (e.g., sensors) may be fabricated. In a specific example, a ring 104 with a single temperature sensor 240 (or other sensor), a power source, and device electronics configured to read the single temperature sensor 240 (or other sensor) may be fabricated. In another specific example, a temperature sensor 240 (or other sensor) may be attached to a user's finger (e.g., using a clamps, spring loaded clamps, etc.). In this case, the sensor may be wired to another computing device, such as a wrist worn computing device that reads the temperature sensor 240 (or other sensor). In other examples, a ring 104 that includes additional sensors and processing functionality may be fabricated.

The housing 205 may include one or more housing 205 components. The housing 205 may include an outer housing 205-b component (e.g., a shell) and an inner housing 205-a component (e.g., a molding). The housing 205 may include additional components (e.g., additional layers) not explicitly illustrated in FIG. 2. For example, in some implementations, the ring 104 may include one or more insulating layers that electrically insulate the device electronics and other conductive materials (e.g., electrical traces) from the outer housing 205-b (e.g., a metal outer housing 205-b). The housing 205 may provide structural support for the device electronics, battery 210, substrate(s), and other components. For example, the housing 205 may protect the device electronics, battery 210, and substrate(s) from mechanical forces, such as pressure and impacts. The housing 205 may also protect the device electronics, battery 210, and substrate(s) from water and/or other chemicals.

The outer housing 205-b may be fabricated from one or more materials. In some implementations, the outer housing 205-b may include a metal, such as titanium, that may provide strength and abrasion resistance at a relatively light weight. The outer housing 205-b may also be fabricated from other materials, such polymers. In some implementations, the outer housing 205-b may be protective as well as decorative.

The inner housing 205-a may be configured to interface with the user's finger. The inner housing 205-a may be formed from a polymer (e.g., a medical grade polymer) or other material. In some implementations, the inner housing 205-a may be transparent. For example, the inner housing 205-a may be transparent to light emitted by the PPG light emitting diodes (LEDs). In some implementations, the inner housing 205-a component may be molded onto the outer housing 205-b. For example, the inner housing 205-a may include a polymer that is molded (e.g., injection molded) to fit into an outer housing 205-b metallic shell.

The ring 104 may include one or more substrates (not illustrated). The device electronics and battery 210 may be included on the one or more substrates. For example, the device electronics and battery 210 may be mounted on one or more substrates. Example substrates may include one or more printed circuit boards (PCBs), such as flexible PCB (e.g., polyimide). In some implementations, the electronics/battery 210 may include surface mounted devices (e.g., surface-mount technology (SMT) devices) on a flexible PCB. In some implementations, the one or more substrates (e.g., one or more flexible PCBs) may include electrical traces that provide electrical communication between device electronics. The electrical traces may also connect the battery 210 to the device electronics.

The device electronics, battery 210, and substrates may be arranged in the ring 104 in a variety of ways. In some implementations, one substrate that includes device electronics may be mounted along the bottom of the ring 104 (e.g., the bottom half), such that the sensors (e.g., PPG system 235, temperature sensors 240, motion sensors 245, and other sensors) interface with the underside of the user's finger. In these implementations, the battery 210 may be included along the top portion of the ring 104 (e.g., on another substrate).

The various components/modules of the ring 104 represent functionality (e.g., circuits and other components) that may be included in the ring 104. Modules may include any discrete and/or integrated electronic circuit components that implement analog and/or digital circuits capable of producing the functions attributed to the modules herein. For example, the modules may include analog circuits (e.g., amplification circuits, filtering circuits, analog/digital conversion circuits, and/or other signal conditioning circuits). The modules may also include digital circuits (e.g., combinational or sequential logic circuits, memory circuits etc.).

The memory 215 (memory module) of the ring 104 may include any volatile, non-volatile, magnetic, or electrical media, such as a random access memory (RAM), read-only memory (ROM), non-volatile RAM (NVRAM), electrically-erasable programmable ROM (EEPROM), flash memory, or any other memory device. The memory 215 may store any of the data described herein. For example, the memory 215 may be configured to store data (e.g., motion data, temperature data, PPG data) collected by the respective sensors and PPG system 235. Furthermore, memory 215 may include instructions that, when executed by one or more processing circuits, cause the modules to perform various functions attributed to the modules herein. The device electronics of the ring 104 described herein are only example device electronics. As such, the types of electronic components used to implement the device electronics may vary based on design considerations.

The functions attributed to the modules of the ring 104 described herein may be embodied as one or more processors, hardware, firmware, software, or any combination thereof. Depiction of different features as modules is intended to highlight different functional aspects and does not necessarily imply that such modules must be realized by separate hardware/software components. Rather, functionality associated with one or more modules may be performed by separate hardware/software components or integrated within common hardware/software components.

The processing module 230-a of the ring 104 may include one or more processors (e.g., processing units), microcontrollers, digital signal processors, systems on a chip (SOCs), and/or other processing devices. The processing module 230-a communicates with the modules included in the ring 104. For example, the processing module 230-a may transmit/receive data to/from the modules and other components of the ring 104, such as the sensors. As described herein, the modules may be implemented by various circuit components. Accordingly, the modules may also be referred to as circuits (e.g., a communication circuit and power circuit).

The processing module 230-a may communicate with the memory 215. The memory 215 may include computer-readable instructions that, when executed by the processing module 230-a, cause the processing module 230-a to perform the various functions attributed to the processing module 230-a herein. In some implementations, the processing module 230-a (e.g., a microcontroller) may include additional features associated with other modules, such as communication functionality provided by the communication module 220-a (e.g., an integrated Bluetooth Low Energy transceiver) and/or additional onboard memory 215.

The communication module 220-a may include circuits that provide wireless and/or wired communication with the user device 106 (e.g., communication module 220-b of the user device 106). In some implementations, the communication modules 220-a, 220-b may include wireless communication circuits, such as Bluetooth circuits and/or Wi-Fi circuits. In some implementations, the communication modules 220-a, 220-b can include wired communication circuits, such as Universal Serial Bus (USB) communication circuits. Using the communication module 220-a, the ring 104 and the user device 106 may be configured to communicate with each other. The processing module 230-a of the ring may be configured to transmit/receive data to/from the user device 106 via the communication module 220-a. Example data may include, but is not limited to, motion data, temperature data, pulse waveforms, heart rate data, HRV data, PPG data, and status updates (e.g., charging status, battery charge level, and/or ring 104 configuration settings). The processing module 230-a of the ring may also be configured to receive updates (e.g., software/firmware updates) and data from the user device 106.

The ring 104 may include a battery 210 (e.g., a rechargeable battery 210). An example battery 210 may include a Lithium-Ion or Lithium-Polymer type battery 210, although a variety of battery 210 options are possible. The battery 210 may be wirelessly charged. In some implementations, the ring 104 may include a power source other than the battery 210, such as a capacitor. The power source (e.g., battery 210 or capacitor) may have a curved geometry that matches the curve of the ring 104. In some aspects, a charger or other power source may include additional sensors that may be used to collect data in addition to, or that supplements, data collected by the ring 104 itself. Moreover, a charger or other power source for the ring 104 may function as a user device 106, in such case the charger or other power source for the ring 104 may be configured to receive data from the ring 104, store and/or process data received from the ring 104, and communicate data between the ring 104 and the servers 110.

In some aspects, the ring 104 includes a power module 225 that may control charging of the battery 210. For example, the power module 225 may interface with an external wireless charger that charges the battery 210 when interfaced with the ring 104. The charger may include a datum structure that mates with a ring 104 datum structure to create a specified orientation with the ring 104 during 104 charging. The power module 225 may also regulate voltage(s) of the device electronics, regulate power output to the device electronics, and monitor the state of charge of the battery 210. In some implementations, the battery 210 may include a protection circuit module (PCM) that protects the battery 210 from high current discharge, over voltage during 104 charging, and under voltage during 104 discharge. The power module 225 may also include electro-static discharge (ESD) protection.

The one or more temperature sensors 240 may be electrically coupled to the processing module 230-a. The temperature sensor 240 may be configured to generate a temperature signal (e.g., temperature data) that indicates a temperature read or sensed by the temperature sensor 240. The processing module 230-a may determine a temperature of the user in the location of the temperature sensor 240. For example, in the ring 104, temperature data generated by the temperature sensor 240 may indicate a temperature of a user at the user's finger (e.g., skin temperature). In some implementations, the temperature sensor 240 may contact the user's skin. In other implementations, a portion of the housing 205 (e.g., the inner housing 205-a) may form a barrier (e.g., a thin, thermally conductive barrier) between the temperature sensor 240 and the user's skin. In some implementations, portions of the ring 104 configured to contact the user's finger may have thermally conductive portions and thermally insulative portions. The thermally conductive portions may conduct heat from the user's finger to the temperature sensors 240. The thermally insulative portions may insulate portions of the ring 104 (e.g., the temperature sensor 240) from ambient temperature.

In some implementations, the temperature sensor 240 may generate a digital signal (e.g., temperature data) that the processing module 230-a may use to determine the temperature. As another example, in cases where the temperature sensor 240 includes a passive sensor, the processing module 230-a (or a temperature sensor 240 module) may measure a current/voltage generated by the temperature sensor 240 and determine the temperature based on the measured current/voltage. Example temperature sensors 240 may include a thermistor, such as a negative temperature coefficient (NTC) thermistor, or other types of sensors including resistors, transistors, diodes, and/or other electrical/electronic components.

The processing module 230-a may sample the user's temperature over time. For example, the processing module 230-a may sample the user's temperature according to a sampling rate. An example sampling rate may include one sample per second, although the processing module 230-a may be configured to sample the temperature signal at other sampling rates that are higher or lower than one sample per second. In some implementations, the processing module 230-a may sample the user's temperature continuously throughout the day and night. Sampling at a sufficient rate (e.g., one sample per second) throughout the day may provide sufficient temperature data for analysis described herein.

The processing module 230-a may store the sampled temperature data in memory 215. In some implementations, the processing module 230-a may process the sampled temperature data. For example, the processing module 230-a may determine average temperature values over a period of time. In one example, the processing module 230-a may determine an average temperature value each minute by summing all temperature values collected over the minute and dividing by the number of samples over the minute. In a specific example where the temperature is sampled at one sample per second, the average temperature may be a sum of all sampled temperatures for one minute divided by sixty seconds. The memory 215 may store the average temperature values over time. In some implementations, the memory 215 may store average temperatures (e.g., one per minute) instead of sampled temperatures in order to conserve memory 215.

The sampling rate, that may be stored in memory 215, may be configurable. In some implementations, the sampling rate may be the same throughout the day and night. In other implementations, the sampling rate may be changed throughout the day/night. In some implementations, the ring 104 may filter/reject temperature readings, such as large spikes in temperature that are not indicative of physiological changes (e.g., a temperature spike from a hot shower). In some implementations, the ring 104 may filter/reject temperature readings that may not be reliable due to other factors, such as excessive motion during 104 exercise (e.g., as indicated by a motion sensor 245).

The ring 104 (e.g., communication module) may transmit the sampled and/or average temperature data to the user device 106 for storage and/or further processing. The user device 106 may transfer the sampled and/or average temperature data to the server 110 for storage and/or further processing.

Although the ring 104 is illustrated as including a single temperature sensor 240, the ring 104 may include multiple temperature sensors 240 in one or more locations, such as arranged along the inner housing 205-a near the user's finger. In some implementations, the temperature sensors 240 may be stand-alone temperature sensors 240. Additionally, or alternatively, one or more temperature sensors 240 may be included with other components (e.g., packaged with other components), such as with the accelerometer and/or processor.

The processing module 230-a may acquire and process data from multiple temperature sensors 240 in a similar manner described with respect to a single temperature sensor 240. For example, the processing module 230 may individually sample, average, and store temperature data from each of the multiple temperature sensors 240. In other examples, the processing module 230-a may sample the sensors at different rates and average/store different values for the different sensors. In some implementations, the processing module 230-a may be configured to determine a single temperature based on the average of two or more temperatures determined by two or more temperature sensors 240 in different locations on the finger.

The temperature sensors 240 on the ring 104 may acquire distal temperatures at the user's finger (e.g., any finger). For example, one or more temperature sensors 240 on the ring 104 may acquire a user's temperature from the underside of a finger or at a different location on the finger. In some implementations, the ring 104 may continuously acquire distal temperature (e.g., at a sampling rate). Although distal temperature measured by a ring 104 at the finger is described herein, other devices may measure temperature at the same/different locations. In some cases, the distal temperature measured at a user's finger may differ from the temperature measured at a user's wrist or other external body location. Additionally, the distal temperature measured at a user's finger (e.g., a “shell” temperature) may differ from the user's core temperature. As such, the ring 104 may provide a useful temperature signal that may not be acquired at other internal/external locations of the body. In some cases, continuous temperature measurement at the finger may capture temperature fluctuations (e.g., small or large fluctuations) that may not be evident in core temperature. For example, continuous temperature measurement at the finger may capture minute-to-minute or hour-to-hour temperature fluctuations that provide additional insight that may not be provided by other temperature measurements elsewhere in the body.

The ring 104 may include a PPG system 235. The PPG system 235 may include one or more optical transmitters that transmit light. The PPG system 235 may also include one or more optical receivers that receive light transmitted by the one or more optical transmitters. An optical receiver may generate a signal (hereinafter “PPG” signal) that indicates an amount of light received by the optical receiver. The optical transmitters may illuminate a region of the user's finger. The PPG signal generated by the PPG system 235 may indicate the perfusion of blood in the illuminated region. For example, the PPG signal may indicate blood volume changes in the illuminated region caused by a user's pulse pressure. The processing module 230-a may sample the PPG signal and determine a user's pulse waveform based on the PPG signal. The processing module 230-a may determine a variety of physiological parameters based on the user's pulse waveform, such as a user's respiratory rate, heart rate, HRV, oxygen saturation, and other circulatory parameters.

In some implementations, the PPG system 235 may be configured as a reflective PPG system 235 where the optical receiver(s) receive transmitted light that is reflected through the region of the user's finger. In some implementations, the PPG system 235 may be configured as a transmissive PPG system 235 where the optical transmitter(s) and optical receiver(s) are arranged opposite to one another, such that light is transmitted directly through a portion of the user's finger to the optical receiver(s).

The number and ratio of transmitters and receivers included in the PPG system 235 may vary. Example optical transmitters may include light-emitting diodes (LEDs). The optical transmitters may transmit light in the infrared spectrum and/or other spectrums. Example optical receivers may include, but are not limited to, photosensors, phototransistors, and photodiodes. The optical receivers may be configured to generate PPG signals in response to the wavelengths received from the optical transmitters. The location of the transmitters and receivers may vary. Additionally, a single device may include reflective and/or transmissive PPG systems 235.

The PPG system 235 illustrated in FIG. 2 may include a reflective PPG system 235 in some implementations. In these implementations, the PPG system 235 may include a centrally located optical receiver (e.g., at the bottom of the ring 104) and two optical transmitters located on each side of the optical receiver. In this implementation, the PPG system 235 (e.g., optical receiver) may generate the PPG signal based on light received from one or both of the optical transmitters. In other implementations, other placements, combinations, and/or configurations of one or more optical transmitters and/or optical receivers are contemplated.

The processing module 230-a may control one or both of the optical transmitters to transmit light while sampling the PPG signal generated by the optical receiver. In some implementations, the processing module 230-a may cause the optical transmitter with the stronger received signal to transmit light while sampling the PPG signal generated by the optical receiver. For example, the selected optical transmitter may continuously emit light while the PPG signal is sampled at a sampling rate (e.g., 250 Hz).

Sampling the PPG signal generated by the PPG system 235 may result in a pulse waveform that may be referred to as a “PPG.” The pulse waveform may indicate blood pressure vs time for multiple cardiac cycles. The pulse waveform may include peaks that indicate cardiac cycles. Additionally, the pulse waveform may include respiratory induced variations that may be used to determine respiration rate. The processing module 230-a may store the pulse waveform in memory 215 in some implementations. The processing module 230-a may process the pulse waveform as it is generated and/or from memory 215 to determine user physiological parameters described herein.

The processing module 230-a may determine the user's heart rate based on the pulse waveform. For example, the processing module 230-a may determine heart rate (e.g., in beats per minute) based on the time between peaks in the pulse waveform. The time between peaks may be referred to as an interbeat interval (IBI). The processing module 230-a may store the determined heart rate values and IBI values in memory 215.

The processing module 230-a may determine HRV over time. For example, the processing module 230-a may determine HRV based on the variation in the IBIs. The processing module 230-a may store the HRV values over time in the memory 215. Moreover, the processing module 230-a may determine the user's respiratory rate over time. For example, the processing module 230-a may determine respiratory rate based on frequency modulation, amplitude modulation, or baseline modulation of the user's IBI values over a period of time. Respiratory rate may be calculated in breaths per minute or as another breathing rate (e.g., breaths per 30 seconds). The processing module 230-a may store user respiratory rate values over time in the memory 215.

The ring 104 may include one or more motion sensors 245, such as one or more accelerometers (e.g., 6-D accelerometers) and/or one or more gyroscopes (gyros). The motion sensors 245 may generate motion signals that indicate motion of the sensors. For example, the ring 104 may include one or more accelerometers that generate acceleration signals that indicate acceleration of the accelerometers. As another example, the ring 104 may include one or more gyro sensors that generate gyro signals that indicate angular motion (e.g., angular velocity) and/or changes in orientation. The motion sensors 245 may be included in one or more sensor packages. An example accelerometer/gyro sensor is a Bosch BMl160 inertial micro electro-mechanical system (MEMS) sensor that may measure angular rates and accelerations in three perpendicular axes.

The processing module 230-a may sample the motion signals at a sampling rate (e.g., 50 Hz) and determine the motion of the ring 104 based on the sampled motion signals. For example, the processing module 230-a may sample acceleration signals to determine acceleration of the ring 104. As another example, the processing module 230-a may sample a gyro signal to determine angular motion. In some implementations, the processing module 230-a may store motion data in memory 215. Motion data may include sampled motion data as well as motion data that is calculated based on the sampled motion signals (e.g., acceleration and angular values).

The ring 104 may store a variety of data described herein. For example, the ring 104 may store temperature data, such as raw sampled temperature data and calculated temperature data (e.g., average temperatures). As another example, the ring 104 may store PPG signal data, such as pulse waveforms and data calculated based on the pulse waveforms (e.g., heart rate values, IBI values, HRV values, and respiratory rate values). The ring 104 may also store motion data, such as sampled motion data that indicates linear and angular motion.

The ring 104, or other computing device, may calculate and store additional values based on the sampled/calculated physiological data. For example, the processing module 230 may calculate and store various metrics, such as sleep metrics (e.g., a Sleep Score), activity metrics, and readiness metrics. In some implementations, additional values/metrics may be referred to as “derived values.” The ring 104, or other computing/wearable device, may calculate a variety of values/metrics with respect to motion. Example derived values for motion data may include, but are not limited to, motion count values, regularity values, intensity values, metabolic equivalence of task values (METs), and orientation values. Motion counts, regularity values, intensity values, and METs may indicate an amount of user motion (e.g., velocity/acceleration) over time. Orientation values may indicate how the ring 104 is oriented on the user's finger and if the ring 104 is worn on the left hand or right hand.

In some implementations, motion counts and regularity values may be determined by counting a number of acceleration peaks within one or more periods of time (e.g., one or more 30 second to 1 minute periods). Intensity values may indicate a number of movements and the associated intensity (e.g., acceleration values) of the movements. The intensity values may be categorized as low, medium, and high, depending on associated threshold acceleration values. METs may be determined based on the intensity of movements during a period of time (e.g., 30 seconds), the regularity/irregularity of the movements, and the number of movements associated with the different intensities.

In some implementations, the processing module 230-a may compress the data stored in memory 215. For example, the processing module 230-a may delete sampled data after making calculations based on the sampled data. As another example, the processing module 230-a may average data over longer periods of time in order to reduce the number of stored values. In a specific example, if average temperatures for a user over one minute are stored in memory 215, the processing module 230-a may calculate average temperatures over a five minute time period for storage, and then subsequently erase the one minute average temperature data. The processing module 230-a may compress data based on a variety of factors, such as the total amount of used/available memory 215 and/or an elapsed time since the ring 104 last transmitted the data to the user device 106.

Although a user's physiological parameters may be measured by sensors included on a ring 104, other devices may measure a user's physiological parameters. For example, although a user's temperature may be measured by a temperature sensor 240 included in a ring 104, other devices may measure a user's temperature. In some examples, other wearable devices (e.g., wrist devices) may include sensors that measure user physiological parameters. Additionally, medical devices, such as external medical devices (e.g., wearable medical devices) and/or implantable medical devices, may measure a user's physiological parameters. One or more sensors on any type of computing device may be used to implement the techniques described herein.

The physiological measurements may be taken continuously throughout the day and/or night. In some implementations, the physiological measurements may be taken during 104 portions of the day and/or portions of the night. In some implementations, the physiological measurements may be taken in response to determining that the user is in a specific state, such as an active state, resting state, and/or a sleeping state. For example, the ring 104 can make physiological measurements in a resting/sleep state in order to acquire cleaner physiological signals. In one example, the ring 104 or other device/system may detect when a user is resting and/or sleeping and acquire physiological parameters (e.g., temperature) for that detected state. The devices/systems may use the resting/sleep physiological data and/or other data when the user is in other states in order to implement the techniques of the present disclosure.

In some implementations, as described previously herein, the ring 104 may be configured to collect, store, and/or process data, and may transfer any of the data described herein to the user device 106 for storage and/or processing. In some aspects, the user device 106 includes a wearable application 250, an operating system (OS), a web browser application (e.g., web browser 280), one or more additional applications, and a GUI 275. The user device 106 may further include other modules and components, including sensors, audio devices, haptic feedback devices, and the like. The wearable application 250 may include an example of an application (e.g., “app”) that may be installed on the user device 106. The wearable application 250 may be configured to acquire data from the ring 104, store the acquired data, and process the acquired data as described herein. For example, the wearable application 250 may include a user interface (UI) module 255, an acquisition module 260, a processing module 230-b, a communication module 220-b, and a storage module (e.g., database 265) configured to store application data.

The various data processing operations described herein may be performed by the ring 104, the user device 106, the servers 110, or any combination thereof. For example, in some cases, data collected by the ring 104 may be pre-processed and transmitted to the user device 106. In this example, the user device 106 may perform some data processing operations on the received data, may transmit the data to the servers 110 for data processing, or both. For instance, in some cases, the user device 106 may perform processing operations that require relatively low processing power and/or operations that require a relatively low latency, whereas the user device 106 may transmit the data to the servers 110 for processing operations that require relatively high processing power and/or operations that may allow relatively higher latency.

In some aspects, the ring 104, user device 106, and server 110 of the system 200 may be configured to evaluate sleep patterns for a user. In particular, the respective components of the system 200 may be used to collect data from a user via the ring 104, and generate one or more scores (e.g., Sleep Score, Readiness Score) for the user based on the collected data. For example, as noted previously herein, the ring 104 of the system 200 may be worn by a user to collect data from the user, including temperature, heart rate, HRV, and the like. Data collected by the ring 104 may be used to determine when the user is asleep in order to evaluate the user's sleep for a given “sleep day.” In some aspects, scores may be calculated for the user for each respective sleep day, such that a first sleep day is associated with a first set of scores, and a second sleep day is associated with a second set of scores. Scores may be calculated for each respective sleep day based on data collected by the ring 104 during the respective sleep day. Scores may include, but are not limited to, Sleep Scores, Readiness Scores, and the like.

In some cases, “sleep days” may align with the traditional calendar days, such that a given sleep day runs from midnight to midnight of the respective calendar day. In other cases, sleep days may be offset relative to calendar days. For example, sleep day's may run from 6:00 pm (18:00) of a calendar day until 6:00 pm (18:00) of the subsequent calendar day. In this example, 6:00 pm may serve as a “cut-off time,” where data collected from the user before 6:00 pm is counted for the current sleep day, and data collected from the user after 6:00 pm is counted for the subsequent sleep day. Due to the fact that most individuals sleep the most at night, offsetting sleep days relative to calendar days may enable the system 200 to evaluate sleep patterns for users in such a manner that is consistent with their sleep schedules. In some cases, users may be able to selectively adjust (e.g., via the GUI) a timing of sleep days relative to calendar days so that the sleep days are aligned with the duration of time that the respective users typically sleep.

In some implementations, each overall score for a user for each respective day (e.g., Sleep Score, Readiness Score) may be determined/calculated based on one or more “contributors,” “factors,” or “contributing factors.” For example, a user's overall Sleep Score may be calculated based on a set of contributors, including: total sleep, efficiency, restfulness, REM sleep, deep sleep, latency, timing, or any combination thereof. The Sleep Score may include any quantity of contributors. The “total sleep” contributor may refer to the sum of all sleep periods of the sleep day. The “efficiency” contributor may reflect the percentage of time spent asleep compared to time spent awake while in bed, and may be calculated using the efficiency average of long sleep periods (e.g., primary sleep period) of the sleep day, weighted by a duration of each sleep period. The “restfulness” contributor may indicate how restful the user's sleep is, and may be calculated using the average of all sleep periods of the sleep day, weighted by a duration of each period. The restfulness contributor may be based on a “wake up count” (e.g., sum of all the wake-ups (when user wakes up) detected during different sleep periods), excessive movement, and a “got up count” (e.g., sum of all the got-ups (when user gets out of bed) detected during the different sleep periods).

The “REM sleep” contributor may refer to a sum total of REM sleep durations across all sleep periods of the sleep day including REM sleep. Similarly, the “deep sleep” contributor may refer to a sum total of deep sleep durations across all sleep periods of the sleep day including deep sleep. The “latency” contributor may signify how long (e.g., average, median, longest) the user takes to go to sleep, and may be calculated using the average of long sleep periods throughout the sleep day, weighted by a duration of each period and the number of such periods (e.g., consolidation of a given sleep stage or sleep stages may be its own contributor or weight other contributors). Lastly, the “timing” contributor may refer to a relative timing of sleep periods within the sleep day and/or calendar day, and may be calculated using the average of all sleep periods of the sleep day, weighted by a duration of each period.

By way of another example, a user's overall Readiness Score may be calculated based on a set of contributors, including: sleep, sleep balance, heart rate, HRV balance, recovery index, temperature, activity, activity balance, or any combination thereof. The Readiness Score may include any quantity of contributors. The “sleep” contributor may refer to the combined Sleep Score of all sleep periods within the sleep day. The “sleep balance” contributor may refer to a cumulative duration of all sleep periods within the sleep day. In particular, sleep balance may indicate to a user whether the sleep that the user has been getting over some duration of time (e.g., the past two weeks) is in balance with the user's needs. Typically, adults need 7-9 hours of sleep a night to stay healthy, alert, and to perform at their best both mentally and physically. However, it is normal to have an occasional night of bad sleep, so the sleep balance contributor takes into account long-term sleep patterns to determine whether each user's sleep needs are being met. The “resting heart rate” contributor may indicate a lowest heart rate from the longest sleep period of the sleep day (e.g., primary sleep period) and/or the lowest heart rate from naps occurring after the primary sleep period.

Continuing with reference to the “contributors” (e.g., factors, contributing factors) of the Readiness Score, the “HRV balance” contributor may indicate a highest HRV average from the primary sleep period and the naps happening after the primary sleep period. The HRV balance contributor may help users keep track of their recovery status by comparing their HRV trend over a first time period (e.g., two weeks) to an average HRV over some second, longer time period (e.g., three months). The “recovery index” contributor may be calculated based on the longest sleep period. Recovery index measures how long it takes for a user's resting heart rate to stabilize during the night. A sign of a very good recovery is that the user's resting heart rate stabilizes during the first half of the night, at least six hours before the user wakes up, leaving the body time to recover for the next day. The “body temperature” contributor may be calculated based on the longest sleep period (e.g., primary sleep period) or based on a nap happening after the longest sleep period if the user's highest temperature during the nap is at least 0.5° C. higher than the highest temperature during the longest period. In some aspects, the ring may measure a user's body temperature while the user is asleep, and the system 200 may display the user's average temperature relative to the user's baseline temperature. If a user's body temperature is outside of their normal range (e.g., clearly above or below 0.0), the body temperature contributor may be highlighted (e.g., go to a “Pay attention” state) or otherwise generate an alert for the user.

In some aspects, the system 200 may support techniques for generation of forward-looking health-related predictions. In particular, the system 100 may utilize historical physiological data collected from a user 102 via a wearable device 104, such as the ring 104, to make predictions regarding the physiological data associated with the user 102 in the future. For example, the system 200 may take the user's physiological data from the current day (e.g., activity data, HRV data, heart rate data, etc.), and predict what the user's Sleep Score will be for the next day. In such cases, the ring 104 may collect physiological data associated with the user 102 via the PPG system 335, the temperature sensors 240, the motion sensors 245, or any combination thereof.

In some cases, the system 200 may utilize a machine learning model (e.g., run on a server 110 or the user device 106 via the processing module 230-b) to make such predictions, and may leverage data collected from other, similar users 102 to improve predictions. For example, the system 200 may collect first physiological data associated with the user 102 via the ring 104 during a first time interval, such as a previous night, to generate a first value of a health-related metric associated with the user 102-b, such as a Sleep Score of the user 102-b. In other words, the system 100 may predict what the user's Sleep Score will be for the following day based on the user's previous physiological data collected during the previous 24 hours (or some other time period). Additionally, the system 100 may predict how the Sleep Score for the following day is expected to change based on hypothetical/expected user actions that the user may engage in at some time in the future (and/or expected or anticipated emotions or other states of being that the user is expected to experience at some point in the future). For example, the system 100 may predict that the user's Sleep Score for the following day will increase by 2 points if the user goes to bed tonight one hour earlier than their normal bedtime. Additionally, the prediction of the second value of the health-related metric (e.g., predicted Sleep Score) may be displayed on the user device 106 via the GUI 275.

In some aspects, the data collected prior to the time period of the predicted values (e.g., prior to the following morning associated with the predicted Sleep Score) may be input into the machine learning model to generate or update the prediction of the second value of the health-related metric. In other words, the system 200 may predict that the user's Sleep Score will increase from an 89 to a 91 if the user goes to bed tonight an hour earlier than normal. If the user subsequently goes for a run after dinner and prior to going to bed, the system 200 may update the predicted Sleep Score for the next morning based on the detected run (e.g., the system 200 may predict that the user's Sleep Score will increase from an 89 to a 93 based on the detected run, and assuming that the user goes to bed an hour earlier than normal).

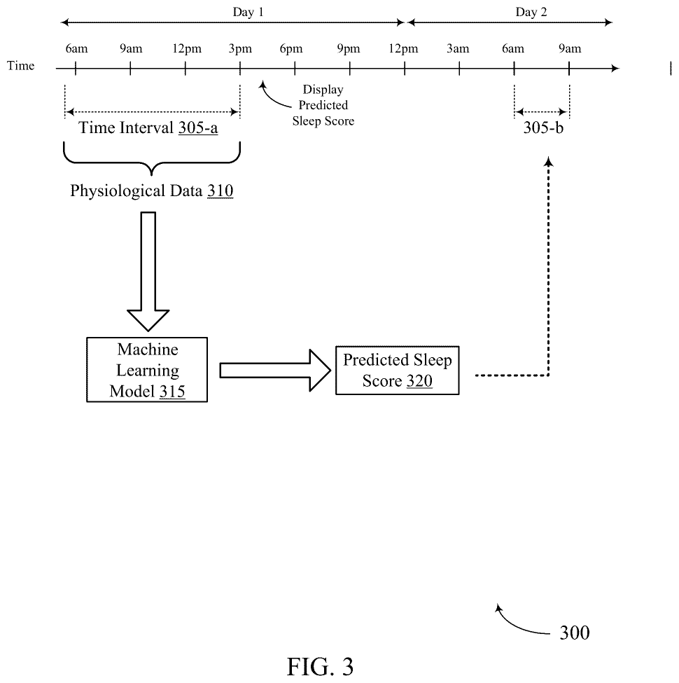

FIG. 3 shows an example of a timeline 300 that supports forward looking health-related prediction in accordance with aspects of the present disclosure. The timeline 300 may implement, or be implemented by, aspects of the system 100, the system 200, or any combination thereof.

In some aspects, the timeline 300 may support techniques for generation of forward-looking health-related predictions. In particular, a wearable device 104 (e.g., wearable ring device 104) associated with a user 102 may collect physiological data 310 during a first time interval 305-a. As shown in FIG. 3, the first time interval 305-a may include a time interval during a current day (e.g., Day 1). The first time interval 305-a may generally span any length of time or day's (e.g., four hours, 30 hours, 1 week, etc.).

The system associated with the wearable device 104 may input the physiological data 310 into a machine learning model 315 to generate a predicted value of a health-related metric for the user 102 at some point in the future, such as a second time interval 305-b. For instance, as shown in FIG. 2, the machine learning model 315 may utilize the physiological data 310 acquired during the first time interval 305-a to generate a predicted Sleep Score for the user 102 for the next morning (e.g., Day 2). In some examples, the machine learning model 315 may utilize additional data to generate the health-related predictions for the second time interval 305-b, such as tags input by the user 102 into a user device 106, one or more activities performed by the user 102, or the like thereof. For instance, if the user tags “caffeine consumption” or “anxiety” in the wearable application 250 executable on the user device 106, such tags may be input into the machine learning model 315 to generate/predict the user's Sleep Score (predicted sleep score 320) for the next morning.

As shown in FIG. 3, the system may display an indication of the predicted Sleep Score 320 to the user 102 via the user device 106. In particular, the system may display predicted values of health-related metrics (e.g., predicted Sleep Scores, predicted heart rates, predicted Readiness Scores, etc.) prior to the time interval that the predictions are expected to occur. In other words, if the predicted values (e.g., predicted Sleep Score 320) is expected to occur or take place during the second time interval 305-b, the system may display the predictions prior to the second time interval 305-b. In some examples, the system may display the indication of the predicted Sleep Score 320 at a given time of day (e.g., 4 pm).

Additionally, the system may modify or adjust the predicted Sleep Score 320 based on one or more hypothetical (e.g., expected, predicted, anticipated) actions to be performed by the user 102, one or more hypothetical/expected tags to be input by the user 102, one or more hypothetical/expected user emotions or states of being, or any combination thereof, between the time interval 305-a and the time interval 305-b. In some examples, the system may predict the one or more hypothetical/expected actions, tags, emotions/states, or being, or any combination thereof, based on a routine of the user 102. That is, the system may identify one or more trends associated with the routine of the user 102 and determine the one or more hypothetical/expected actions, tags, and/or emotions/states of being, based on the one or more trends. For example, the system may identify that the user 102 typically goes to bed between 9 pm and 10 pm and may generate the predicted Sleep Score 320 based on the expected bedtime between 9 pm and 10 pm. That is, the system may input the expected bedtime into the machine learning model 315 to update the predicted Sleep Score 320.

In some aspects, the user's expected future actions and/or expected future emotions or states of being (e.g., relaxation, stress, etc.), may also be predicted based on detected user activity patterns, measured physiological data, and/or based on other contextual factors such as time, location, etc. In some cases, the system (e.g., machine learning model) may calculate probabilities or likelihoods that the expected future actions, emotions, and/or states of being will occur without input from the user, and may use the determined probabilities/likelihoods when making future health-related predictions. For example, if the system detects that the user has a fever, the system may then determine that it is unlikely that the user will participate in their usual basketball league that night (which may be determined based on the user's routine, a calendar application, and/or previous tags input by the user). In this regard, the system may use the physiological data associated with the fever and the expectation that the user will not play basketball that night when making health-related predictions for the user. Additionally, or alternatively, the user 102 may input one or more expected actions to be performed by the user 102, one or more expected tags to be input by the user 102, one or more expected emotions/states of being, or any combination thereof, between the time interval 305-a and the time interval 305-b. For example, at 4 pm the user 102 may input that they intend to have alcohol (e.g., tag alcohol) at 6 pm. As such, the system may modify or adjust the predicted Sleep Score 320 based on the hypothetical, or predicted, consumption of alcohol at 6 pm. That is, the system may input the hypothetical alcohol consumption into the machine learning model 315 to update the predicted Sleep Score 320.

In some examples, the system may request the user 102 to confirm or deny whether they performed the one or more hypothetical (e.g., expected, predicted) actions, input the one or more hypothetical tags, whether they experienced the hypothetical/expected emotions/states of being, or any combination thereof, within a threshold duration of a hypothetical time associated with the one or more hypothetical actions or the one or more hypothetical tags. In such cases, the system may update the predicted Sleep Score 320 based on whether the user 102 confirmed or denied the one or more hypothetical actions, the one or more hypothetical tags, or both. That is, the system may update the predicted Sleep Score 320 based on the user 102 failing to confirm or denying the one or more hypothetical actions, the one or more hypothetical tags, or both. Continuing with the previous example, at 7 pm, the system may prompt the user 102 to confirm or deny consumption of alcohol at 6 pm. In some examples, the user 102 may confirm the alcohol consumption and the system may maintain the predicted Sleep Score 320. Alternatively, the user 102 may deny the alcohol consumption (e.g., or fail to confirm within a threshold duration of prompting the user 102) and the system may update the predicted Sleep Score 320 based on failing to consume alcohol at 6 pm.

By way of another example, the user may go for a run after dinner at 7 pm. In such cases, the wearable device may recognize that the user went for a run, and/or the user may tag the run in the wearable application 250. In such cases, the system may input the physiological data (and/or tagged run) into the machine learning model 315 so that the machine learning model 315 may update the user's predicted Sleep Score 320 for the next morning. In this example, at 7:45 pm, and after completion of the run, the wearable application 250 may display a message that states: “Great run! We expect your Sleep Score tomorrow morning to improve by 2 points due to this added exercise before bed.”

Additionally, or alternatively, the system may confirm or deny whether the user 102 performed the one or more hypothetical/expected actions, input the one or more hypothetical/expected tags, experienced the hypothetical/expected emotions/states of being, or any combination thereof, based on additional physiological data 310. For example, the system may input a hypothetical nap from 3 pm to 4 pm into the machine learning model 315 to generate the predicted Sleep Score 320. As such, at 4 pm, the system may identify, based on additional physiological data 310 collected from 3 pm to 4 pm, whether the user 102 took a nap. As such, the system may confirm or deny the nap based on the additional physiological data 310 and may update the predicted Sleep Score 320 accordingly.

In some aspects, the system may further identify actual values of the health-related metrics during the second time interval 305-b, and may compare the actual values to the predicted values to further train the machine learning model 315. For example, continuing with reference to the timeline 300, the system (e.g., wearable device 104, user device 106, servers 110) may calculate the user's actual Sleep Score after the user 102 wakes up on Day 2. In this example, the system may input the actual Sleep Score into the machine learning model 315 so that the machine learning model 315 is able to compare the user's predicted Sleep Score to the user's actual Sleep Score to improve Sleep Score predictions for the user in the future.

For instance, at 4 pm on Day 1, the system may predict that the user's sleep score will be 92 the next morning. Subsequently, after waking up on Day 2, the system may calculate the user's actual Sleep Score to be 89. In this example, the predicted Sleep Score (92) and the actual Sleep Score (89) may be re-inputted into the machine learning model 315 so that the machine learning model 315 may “learn” or otherwise be trained to recognize what caused the discrepancy between the predicted and actual Sleep Score. In some cases, the system may prompt the user to input additional information that may be used to determine what caused the discrepancy (e.g., “Please confirm that you went to bed at 9:30 pm,” “Did you consume alcohol last night?,” “Was something bothering you during your sleep last night?”).

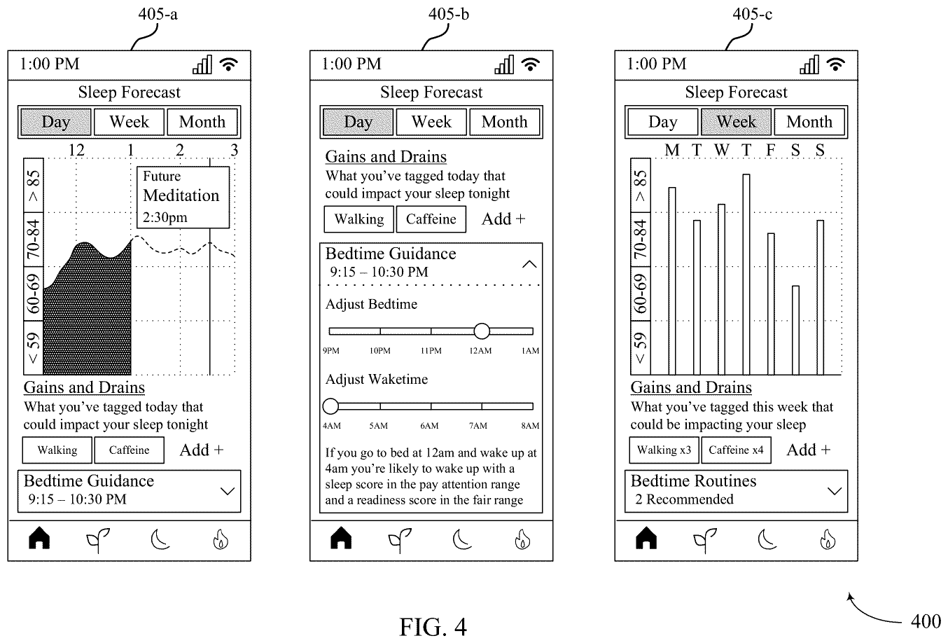

FIG. 4 shows an example of a GUI 400 that supports forward looking health-related prediction in accordance with aspects of the present disclosure. The GUI 400 may implement, or be implemented by, aspects of the system 100, the system 200, the timeline 300, or any combination thereof. In some examples, the GUI 400 may be an example of a GUI of a user device 106, as described with reference to FIGS. 1 and 2. For example, the GUI 400 may be an example of a GUI 275 of a user device 106 as described with reference to FIGS. 1 and 2. In the example of FIG. 4, the GUI 400 may include an application interfaces 405-a, 405-b, and 405-c that may be displayed to a user 102 via the GUI 400.

The application interfaces 405 may be associated with an application (e.g., wearable application 250) running on a user device 106. In some examples, the application interfaces 405 may include a set of graphical elements (also referred to as widgets or components) that the application provides so that a user 102 may provide input to, and receive output from, the application via the application interface 405. In some examples, one or more operations associated with the GUI 400 may be performed based on a manipulation of the one or more graphical elements associated with the GUI 400. Examples of graphical elements associated with the GUI 400 may include, but are not limited to, buttons, sliders, dropdown lists, tabs, text boxes, and the like. The application interface 405 may also include a set of tabs enabling the user 102 to switch between different features of the application. For example, the set of tabs may allow the user 102 to switch between one or more of a “home feature,” a “readiness feature,” a “sleep feature,” or an “activity feature”, in the application running on the user device 106.

In the example of FIG. 4, a system associated with the user device 106 may input data associated with the user 102 into a machine learning model (e.g., machine learning model 315 in FIG. 3) to predict one or more health-related metrics associated with the user 102 (e.g., changes in the one or more health related metrics associated with the user 102). That is, the system may collect first physiological data associated with the user 102 during a first time interval (e.g., time interval 305-a in FIG. 3) to generate a first value of one or more health-related metrics associated with the user 102. Additionally, the system may acquire (e.g., collect) additional physiological data associated with the user 102, as well as one or more tags input into the user device 106 by the user 102, prior to a second time interval (e.g., time interval 305-b in FIG. 3).

As such, the system may input the additional physiological data, the one or more tags, or both, into the machine learning model to predict a second value of the one or more health-related metric during the second time interval (e.g., from the first time interval to the second time interval), which may be referred to as the one or more predicted health-related metrics. In other words, the system may identify one or more first relationships (e.g., trends) between physiological data associated with the user 102, tags recorded by the user 102, or both, and a health-related metric, and adjust one or more parameters of the machine learning model based on the one or more first relationships. As such, the machine learning model may predict changes in the health-related metric based on data associated with the user 102 and the one or more relationships associated with the user 102.

Additionally, or alternatively, the system may identify one or more second relationships between physiological data associated with a group of users 102, tags recorded by the group of users 102, or both, and the health-related metric and adjust the one or more parameters of the machine learning model based on the one or more second relationships. That is, the machine learning model may predict the second value of the one or more health-related metrics based on data associated with the user 102 and the one or more second relationships associated with the group of users 102, such as other users that share similar biographical data, routines, and/or physiological data as the user 102.

For example, the system may input sleep data associated with the user 102 during a previous night, as well as data associated with a circadian rhythm of the user 102, into the machine learning model to predict a Sleep Score and a Readiness Score of the user 102 for the next day. Additionally, the system collect data associated with the user 102 throughout the day (e.g., between the previous night and the following night) and input the collected data into the machine learning model (e.g., as it is collected) to adjust, or modify, the prediction of the Sleep Score, the Readiness Score, or both. For example, the data associated with the user 102 collected throughout the day may include one or more tags (e.g., caffeine, alcohol, etc.) input by the user 102, one or more activities (e.g., workouts, naps, restorative time, etc.) completed by the user 102, physiological data associated with the user 102 (e.g., heart rate, etc.), or any combination thereof. As such, the system may display, via GUI 400, an indication of the predicted Sleep Score, the predicted Readiness Score, or both, for the following day, to the user 102 as the day progresses.

For example, the GUI 400 may display an indication of a sleep forecast to the user 102. The sleep forecast may indicate a predicted Sleep Score of the user 102 throughout a time interval, such as a day, week, month, year, or any combination thereof. Additionally, the sleep forecast may display a history (e.g., record) of the predicted Sleep Score (e.g., at earlier times of the time interval), as well as the predicted Sleep Score through the remainder of the time interval. For example, as depicted in the application interface 405-a, the GUI 400 may display an indication of a sleep forecast of the user 102 throughout a day. In another example, as depicted in an application interface 405-c, the GUI 400 may display an indication of a sleep forecast (e.g., indicating a Sleep Score, predicted or actual) of the user 102 over a week.

Additionally, the GUI 400 may display an indication of one or more tags (e.g., input by the user 102) that may be impacting the predicted Sleep Score of the user 102, such as walking and caffeine. In some examples, the sleep forecast may display an indication of how each of the one or more tags impacted the predicted Sleep Score. For example, the application interface 405-a may display an indication that the user 102 consumed caffeine at 12 pm, which resulted in a decrease in the predicted Sleep Score, and that the user 102 took a walk at 12:40 pm, which resulted in an increase in the predicted Sleep Score.

In some examples, the system may collect, or receive, data associated with the user 102 from one or more applications associated with the wearable device 104, the user device 102, or both, for input into the machine learning model. For example, the one or more applications may include a lifestyle application, a social media application, a utility application, an entertainment application, a productivity application, an information outlet application, a calendar application, or any combination thereof. In other words, the system may input the data associated with the user 102 from the one or more applications into the machine learning model, such that the one or more predicted health-related metrics are based on the data from the one or more applications.

Additionally, or alternatively, the user 102 may input, into the system, one or more hypothetical actions (e.g., activities, tags, etc.) to be performed by the user 102 (e.g., subsequent to the first time interval) into the machine learning model and the system may adjust the one or more predicted health-related metrics based on the hypothetical action. Examples of hypothetical actions may include, but are not limited to, a hypothetical bedtime, a hypothetical waketime, a hypothetical workout, a hypothetical nap, a hypothetical meal consumption, a hypothetical drink (e.g., caffeine, alcohol, etc.) consumption, or the like thereof. For example, as depicted in the application interface 405-a, the user 102 may input, via the user device 106, that the user 102 intends to meditate at 2:40 pm. As such, the system may adjust the predicted Sleep Score of the user 102 based on meditating at 2:40 pm. As described in the previous example, the hypothetical action may be associated with timing information (e.g., as input via the user 102), such that the adjustment to the one or more predicted health-related metrics are based on timing of the hypothetical action. For example, consuming caffeine at 8 am may have minimal impact on a Sleep Score of the user 102 as compared to consuming caffeine at 5 pm.

In another example, as depicted in an application interface 405-b, the GUI 400 may display a slide bar indicating a hypothetical bedtime and a hypothetical waketime. As such, the user 102 may adjust the hypothetical bedtime, the hypothetical waketime, or both, and the system may input the hypothetical bedtime, the hypothetical waketime, or both, into the machine learning model to predict a Sleep Score and a Readiness Score of the user 102 for the next day (e.g., following the hypothetical bedtime and the hypothetical waketime). Thus, the GUI 400 may display information associated with the predicted Sleep Score and Readiness Score to the user 102. As the user slides the slide bars for the hypothetical bedtime and hypothetical waketime, the system may update the predicted Sleep Score and the predicted Readiness Score (in real time, or near-real time) and display the updated predictions so that the user can plan their sleeping schedule accordingly. For example, the user 102 may input a hypothetical bedtime of 12 am and a hypothetical waketime of 4 am and the GUI 400 may display a message indicating “If you go to bed at 12 am, and wake up at 4 am you're likely to wake up with a Sleep Score in the “pay attention” range and a Readiness Score in the “fair” range.

In some examples, the system may input one or more hypothetical actions associated with a routine of the user 102 into the machine learning model to generate the one or more predicted health-related metrics. That is, the system may receive, from the wearable device 104, baseline physiological data measured from the user 102 prior to the first time interval to determine one or more characteristics of the routine of the user 102 (e.g., the user's average bedtime, the user's average waketime, the user's average Sleep Score, etc.). Additionally, or alternatively, the system may receive, from the user device 106, one or more tags recorded from the user 102 prior to the first time interval to determine the one or more characteristics of the routine of the user 102. In such cases, the one or more characteristics of the routine may include a bedtime, a waketime, a workout timing, a meditation timing, a workout type, a meal timing, a nap timing, a nap duration, or any combination thereof. For example, the system may identify that the user 102 typically plays basketball on Thursday evenings at 8 pm that results in their bedtime being 10 pm rather than 9 pm. As such, on Thursday, the system may input a hypothetical bedtime of 10 pm and a hypothetical action of playing basketball at 8 pm into the machine learning model to predict the second value of the one or more health-related metrics of the user 102.

In some examples, the system may recommend, or suggest, one or more actions to pre-empt, adjust, or maintain a predicted change in health-related metric prior to the second time interval (e.g., maintain a predicted health-related metrics). For example, the system may identify that the predicted change in the health-related metric may negatively impact the user 102 (e.g., be below a threshold value). As such, the system may determine one or more recommended actions that the user 102 may perform to reduce the predicted change (e.g., decrease the change in value) in the health-related metric. Additionally, or alternatively, the system may identify that the predicted change in the health-related metric may positively impact the user 102 (e.g., be above a second threshold value). As such, the system may determine one or more recommended actions that the user 102 may perform to maintain the predicted change in the health-related metric.

Additionally, or alternatively, the user 102 may input a desired value of a health-related metric, such that the system may recommend, or suggest, one or more actions to achieve the desired value of the health related metric. For example, the user 102 may input that they would like to have a Sleep Score in the “good” range, however, the system may predict that the user 102 is likely to have a Sleep Score in the “fair” range. As such, the system may identify one or more recommended actions that may improve the predicted Sleep Score of the user 102 from the “fair” range to the “good” range. For example, as depicted in the application interface 405-c, the GUI 400 may display an indication of one or more recommended actions associated with a bedtime routine of the user 102, such as moving a bedtime of the user 102 an hour earlier, limiting screen time an hour before the bedtime of the user 102, or the like thereof. Additionally, the GUI 400 may display an indication of the predicted change in the health-related metric based on performing the one or more recommended actions.

In some examples, the one or more recommended actions may include a modification to an environment (e.g., physical environment) of the user 102. That is, the system may receive sensor data from the wearable device 104, the user device 106, another data source (e.g., charger of the wearable device 104, smart thermostat, smart home assistant device, etc.), associated with one or more characteristics of the environment of the user 102. The system may identify (e.g., based on the one or more first relationships, the one or more second relationships, or both) how the one or more characteristics of the environment impacted the one or more health-related metrics of the user 102 during the first time interval. Additionally, the system may identify how modifying the one or more characteristics of the environment may result in a positive predicted change to the one or more health-related metrics and, as such, may suggest, to the user 102 via the GUI 400, one or more modifications to the one or more characteristics of the environment. For example, the system may identify that, during a previous night, a bedroom that the user 102 slept in was at a temperature of 75 degrees and that the temperature of 75 degrees resulted in an elevated heart rate of the user 102 that further resulted in a negative impact to a Sleep Score of the user 102 during the previous night. As such, the system may display, via the GUI 400, an indication of a recommendation to reduce the temperature of the bedroom to 70 degrees for a following night to improve the Sleep Score of the user 102. In some cases, the system may display how the hypothetical change to the user's environment is expected to change their Sleep Score (or some other physiological parameter) for the following day.

In some examples, the system may update, or adjust, one or more parameters of the machine learning model based on an actual change in the one or more health-related metrics from the first time interval to the second time interval. In other words, the system may collect physiological data associated with the user 102 during the second time interval to generate an actual value of the one or more health-related metrics during the second time interval. Additionally, the system may compare the first value of the one or more health-related metrics during the first time interval to the actual value of the one or more health-related metrics during the second time interval to identify an actual change in a value of the one or more health related metrics. As such, the system may compare the actual value of the one or more health-related metrics to the predicted, or second, value of the one or more health-related metrics to validate (e.g., or invalidate) the one or more first relationships, the one or more second relationships, or both. As such, the system may update the one or more parameters of the machine learning model based on validation of the one or more first relationships, the one or more second relationships, or both.

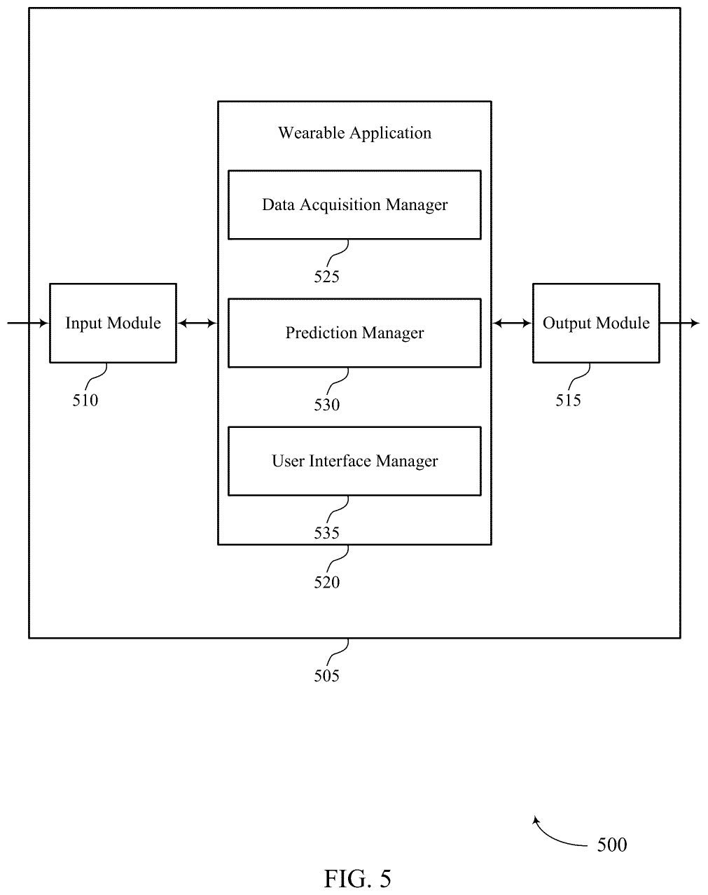

FIG. 5 shows a block diagram 500 of a device 505 that supports forward looking health-related prediction in accordance with aspects of the present disclosure. The device 505 may include an input module 510, an output module 515, and a wearable application 520. The device 505 may also include a processor. Each of these components may be in communication with one another (e.g., via one or more buses).

The input module 510 may provide a means for receiving information such as packets, user data, control information, or any combination thereof associated with various information channels (e.g., control channels, data channels, information channels related to illness detection techniques). Information may be passed on to other components of the device 505. The input module 510 may utilize a single antenna or a set of multiple antennas.

The output module 515 may provide a means for transmitting signals generated by other components of the device 505. For example, the output module 515 may transmit information such as packets, user data, control information, or any combination thereof associated with various information channels (e.g., control channels, data channels, information channels related to illness detection techniques). In some examples, the output module 515 may be co-located with the input module 510 in a transceiver module. The output module 515 may utilize a single antenna or a set of multiple antennas.

For example, the wearable application 520 may include a data acquisition manager 525, a prediction manager 530, a user interface manager 535, or any combination thereof. In some examples, the wearable application 520, or various components thereof, may be configured to perform various operations (e.g., receiving, monitoring, transmitting) using or otherwise in cooperation with the input module 510, the output module 515, or both. For example, the wearable application 520 may receive information from the input module 510, send information to the output module 515, or be integrated in combination with the input module 510, the output module 515, or both to receive information, transmit information, or perform various other operations as described herein.

The wearable application 520 may support generating personalized health-related predictions from measured physiological data in accordance with examples as disclosed herein. The data acquisition manager 525 may be configured as or otherwise support a means for receiving, from a wearable device, first physiological data measured from a user via the wearable device throughout a first time interval. The prediction manager 530 may be configured as or otherwise support a means for outputting, via a machine learning model based at least in part on inputting the first physiological data into the machine learning model, one or more health-related predictions associated with the user during a second time interval, wherein the one or more health-related predictions comprise a predicted change in a health-related metric during the second time interval from a first predicted value to a second predicted value based at least in part on one or more hypothetical user actions engaged in by the user between the first time interval and the second time interval. The user interface manager 535 may be configured as or otherwise support a means for causing a GUI of a user device associated with the wearable device to display information associated with the one or more health-related predictions prior to the second time interval.

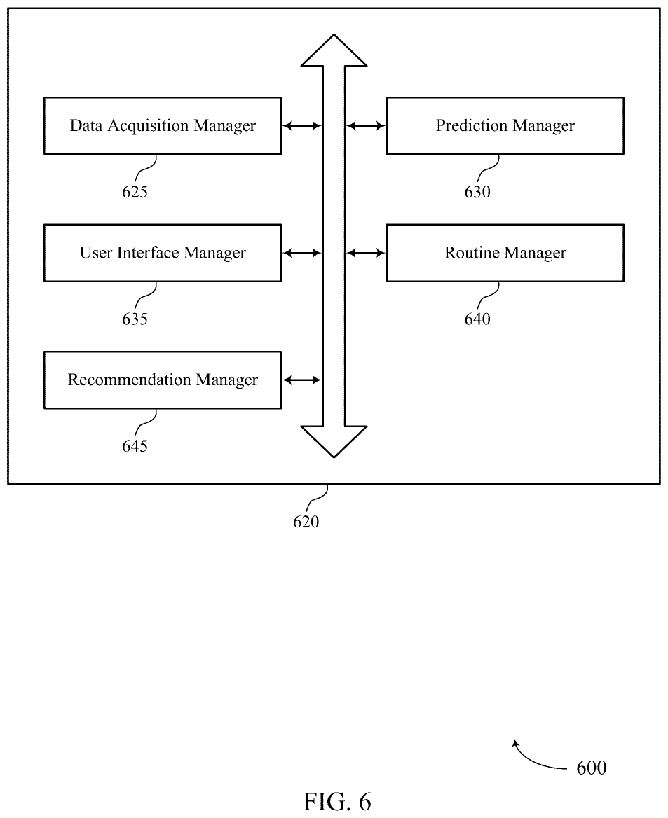

FIG. 6 shows a block diagram 600 of a wearable application 620 that supports forward looking health-related prediction in accordance with aspects of the present disclosure. The wearable application 620 may be an example of aspects of a wearable application or a wearable application 520, or both, as described herein. The wearable application 620, or various components thereof, may be an example of means for performing various aspects of forward looking health-related prediction as described herein. For example, the wearable application 620 may include a data acquisition manager 625, a prediction manager 630, a user interface manager 635, a routine manager 640, a recommendation manager 645, or any combination thereof. Each of these components may communicate, directly or indirectly, with one another (e.g., via one or more buses).

The wearable application 620 may support generating personalized health-related predictions from measured physiological data in accordance with examples as disclosed herein. The data acquisition manager 625 may be configured as or otherwise support a means for receiving, from a wearable device, first physiological data measured from a user via the wearable device throughout a first time interval. The prediction manager 630 may be configured as or otherwise support a means for outputting, via a machine learning model based at least in part on inputting the first physiological data into the machine learning model, one or more health-related predictions associated with the user during a second time interval, wherein the one or more health-related predictions comprise a predicted change in a health-related metric during the second time interval from a first predicted value to a second predicted value based at least in part on one or more hypothetical user actions engaged in by the user between the first time interval and the second time interval. The user interface manager 635 may be configured as or otherwise support a means for causing a GUI of a user device associated with the wearable device to display information associated with the one or more health-related predictions prior to the second time interval.

In some examples, the data acquisition manager 625 may be configured as or otherwise support a means for receiving, via the user device, a user input indicating one or more additional hypothetical user actions to be performed by the user subsequent to the first time interval and prior to the second time interval. In some examples, the prediction manager 630 may be configured as or otherwise support a means for outputting, from the machine learning model based at least in part on inputting the user input to the machine learning model, one or more modifications to the one or more health-related predictions. In some examples, the user interface manager 635 may be configured as or otherwise support a means for causing the GUI of the user device to display additional information associated with the one or more modifications to the one or more health-related predictions.

In some examples, the user input further indicates timing information associated with the one or more additional hypothetical user actions.

In some examples, the one or more additional hypothetical user actions comprise a hypothetical bedtime, a hypothetical wake time, a hypothetical workout, a hypothetical nap, a hypothetical meal consumption, a hypothetical caffeine consumption, a hypothetical alcohol consumption, or any combination thereof.

In some examples, the data acquisition manager 625 may be configured as or otherwise support a means for receiving, from the wearable device, baseline physiological data measured from the user via the wearable device prior to the first time interval. In some examples, the routine manager 640 may be configured as or otherwise support a means for determining one or more characteristics of a routine of the user based at least in part on the baseline physiological data, wherein the one or more hypothetical user actions are based at least in part on the one or more characteristics of the routine of the user.

In some examples, the one or more characteristics of the routine comprise a bedtime, a wake-time, a workout timing, a meditation timing, a workout type, a meal timing, a nap timing, a nap duration, or any combination thereof.

In some examples, the user interface manager 635 may be configured as or otherwise support a means for causing the GUI of the user device to display the one or more hypothetical user actions used to generate the one or more health-related predictions.

In some examples, the data acquisition manager 625 may be configured as or otherwise support a means for receiving, from the wearable device, second physiological data measured from the user via the wearable device subsequent to the first time interval and prior to the second time interval. In some examples, the prediction manager 630 may be configured as or otherwise support a means for outputting, from the machine learning model based at least in part on inputting the second physiological data to the machine learning model, one or more modifications to the one or more health-related predictions. In some examples, the user interface manager 635 may be configured as or otherwise support a means for causing the GUI of the user device to display additional information associated with the one or more modifications to the one or more health-related predictions.

In some examples, the data acquisition manager 625 may be configured as or otherwise support a means for receiving, via the user device, a user input indicating one or more tags associated with the user subsequent to the first time interval and prior to the second time interval. In some examples, the prediction manager 630 may be configured as or otherwise support a means for outputting, from the machine learning model based at least in part on inputting the one or more tags to the machine learning model, one or more modifications to the one or more health-related predictions. In some examples, the user interface manager 635 may be configured as or otherwise support a means for causing the GUI of the user device to display additional information associated with the one or more modifications to the one or more health-related predictions.

In some examples, the recommendation manager 645 may be configured as or otherwise support a means for determining one or more recommended actions to preempt, adjust, or maintain the predicted change in the health-related metric prior to the second time interval. In some examples, the user interface manager 635 may be configured as or otherwise support a means for causing the GUI of the user device associated with the wearable device to display the one or more recommended actions to preempt, adjust, or maintain the predicted change in the health-related metric prior to the second time interval.

In some examples, the data acquisition manager 625 may be configured as or otherwise support a means for receiving sensor data from the wearable device, the user device, or both, the sensor data associated with one or more characteristics of a physical environment of the user during the first time interval, wherein the one or more recommended actions comprise a recommended modification to the one or more characteristics of the physical environment of the user during the second time interval.

In some examples, the data acquisition manager 625 may be configured as or otherwise support a means for receiving, via the user device, a user input indicating a desired value of health-related metric during the second time interval. In some examples, the recommendation manager 645 may be configured as or otherwise support a means for determining one or more recommended actions to achieve the desired value of the health-related metric during the second time interval, wherein the one or more recommended actions are based at least in part on a difference between the second predicted value and the desired value. In some examples, the user interface manager 635 may be configured as or otherwise support a means for causing the GUI of the user device associated with the wearable device to display the one or more recommended actions to achieve the desired value of the health-related metric during the second time interval.

In some examples, the data acquisition manager 625 may be configured as or otherwise support a means for receiving second physiological data measured from the user via the wearable device throughout the time interval. In some examples, the prediction manager 630 may be configured as or otherwise support a means for calculating an actual value of the health-related metric during the second time interval based at least in part on the second physiological data. In some examples, the prediction manager 630 may be configured as or otherwise support a means for adjusting one or more parameters of the machine learning model based at least in part on a comparison between the actual value of the health-related metric during the second time interval and the second predicted value of the health-related metric during the second time interval.

In some examples, the data acquisition manager 625 may be configured as or otherwise support a means for receiving, prior to the second time interval, user data associated with the user from one or more applications associated with the wearable device, the user device, or both, the one or more applications comprising a lifestyle application, a social media application, a utility application, an entertainment application, a productivity application, an information outlet application, or any combination thereof. In some examples, the prediction manager 630 may be configured as or otherwise support a means for inputting the user data into the machine learning model, wherein the one or more health-related predictions associated with the user during the second time interval are based at least in part on inputting the user data to the machine learning model.

In some examples, the predicted change in the health-related metric during the second time interval is based at least in part on timing information associated with the one or more hypothetical user actions engaged in by the user between the first time interval and the second time interval.

In some examples, the machine learning model is trained based at least in part on data associated with the user, data associated with a set of users, or both.

In some examples, the first physiological data measured from the user via the wearable device throughout the first time interval comprises data associated with a Sleep Score of the user, a Readiness Score of the user, or both.

In some examples, the data associated with a Sleep Score of the user comprises data associated with a circadian rhythm of the user.

In some examples, the wearable device comprises a ring wearable device.

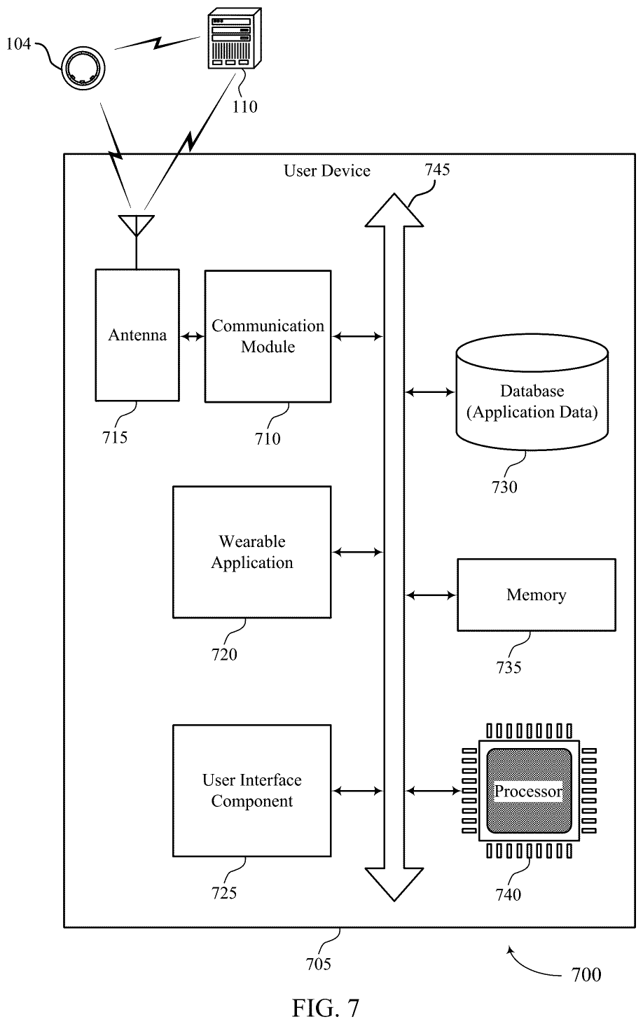

FIG. 7 shows a diagram of a system 700 including a device 705 that supports forward looking health-related prediction in accordance with aspects of the present disclosure. The device 705 may be an example of or include the components of a device 505 as described herein. The device 705 may include an example of a user device 106, as described previously herein. The device 705 may include components for bi-directional communications including components for transmitting and receiving communications with a wearable device 104 and a server 110, such as a wearable application 720, a communication module 710, an antenna 715, a user interface component 725, a database (application data) 730, a memory 735, and a processor 740. These components may be in electronic communication or otherwise coupled (e.g., operatively, communicatively, functionally, electronically, electrically) via one or more buses (e.g., a bus 745).

The communication module 710 may manage input and output signals for the device 705 via the antenna 715. The communication module 710 may include an example of the communication module 220-b of the user device 106 shown and described in FIG. 2. In this regard, the communication module 710 may manage communications with the ring 104 and the server 110, as illustrated in FIG. 2. The communication module 710 may also manage peripherals not integrated into the device 705. In some cases, the communication module 710 may represent a physical connection or port to an external peripheral. In some cases, the communication module 710 may utilize an operating system such as iOS®, ANDROID®, MS-DOS®, MS-WINDOWS®, OS/2®, UNIX®, LINUX®, or another known operating system. In other cases, the communication module 710 may represent or interact with a wearable device (e.g., ring 104), modem, a keyboard, a mouse, a touchscreen, or a similar device. In some cases, the communication module 710 may be implemented as part of the processor 740. In some examples, a user may interact with the device 705 via the communication module 710, user interface component 725, or via hardware components controlled by the communication module 710.

In some cases, the device 705 may include a single antenna 715. However, in some other cases, the device 705 may have more than one antenna 715, that may be capable of concurrently transmitting or receiving multiple wireless transmissions. The communication module 710 may communicate bi-directionally, via the one or more antennas 715, wired, or wireless links as described herein. For example, the communication module 710 may represent a wireless transceiver and may communicate bi-directionally with another wireless transceiver. The communication module 710 may also include a modem to modulate the packets, to provide the modulated packets to one or more antennas 715 for transmission, and to demodulate packets received from the one or more antennas 715.

The user interface component 725 may manage data storage and processing in a database 730. In some cases, a user may interact with the user interface component 725. In other cases, the user interface component 725 may operate automatically without user interaction. The database 730 may be an example of a single database, a distributed database, multiple distributed databases, a data store, a data lake, or an emergency backup database.

The memory 735 may include RAM and ROM. The memory 735 may store computer-readable, computer-executable software including instructions that, when executed, cause the processor 740 to perform various functions described herein. In some cases, the memory 735 may contain, among other things, a BIOS that may control basic hardware or software operation such as the interaction with peripheral components or devices.

The processor 740 may include an intelligent hardware device, (e.g., a general-purpose processor, a DSP, a CPU, a microcontroller, an ASIC, an FPGA, a programmable logic device, a discrete gate or transistor logic component, a discrete hardware component, or any combination thereof). In some cases, the processor 740 may be configured to operate a memory array using a memory controller. In other cases, a memory controller may be integrated into the processor 740. The processor 740 may be configured to execute computer-readable instructions stored in a memory 735 to perform various functions (e.g., functions or tasks supporting a method and system for sleep staging algorithms).

The wearable application 720 may support generating personalized health-related predictions from measured physiological data in accordance with examples as disclosed herein. For example, the wearable application 720 may be configured as or otherwise support a means for receiving, from a wearable device, first physiological data measured from a user via the wearable device throughout a first time interval. The wearable application 720 may be configured as or otherwise support a means for outputting, via a machine learning model based on inputting the first physiological data into the machine learning model, one or more health-related predictions associated with the user during a second time interval, wherein the one or more health-related predictions comprise a predicted change in a health-related metric during the second time interval from a first predicted value to a second predicted value based at least in part on one or more hypothetical user actions engaged in by the user between the first time interval and the second time interval. The wearable application 720 may be configured as or otherwise support a means for causing a GUI of a user device associated with the wearable device to display information associated with the one or more health-related predictions prior to the second time interval.

By including or configuring the wearable application 720 in accordance with examples as described herein, the device 705 may support techniques for generating forward-looking health-related predictions which may improve user experience related to understanding how their choices (e.g., actions) impact their overall health, increase user motivation to proactively make choices to improve their overall health, and increase user engagement with tags, among other advantages.

The wearable application 720 may include an application (e.g., “app”), program, software, or other component that is configured to facilitate communications with a ring 104, server 110, other user devices 106, and the like. For example, the wearable application 720 may include an application executable on a user device 106 that is configured to receive data (e.g., physiological data) from a ring 104, perform processing operations on the received data, transmit and receive data with the servers 110, and cause presentation of data to a user 102.

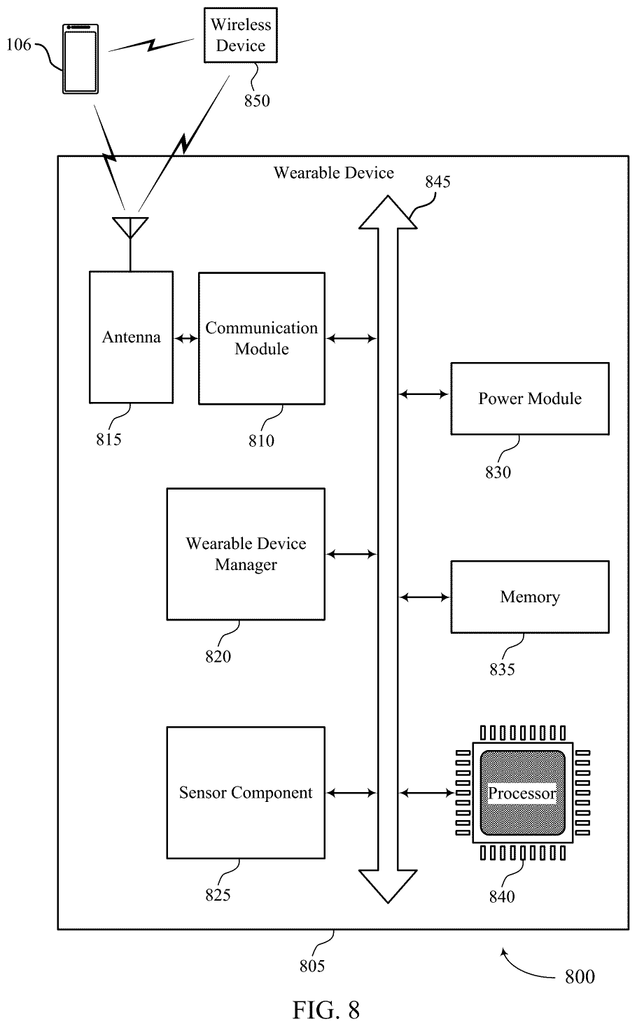

FIG. 8 shows a diagram of a system 800 including a device 805 that supports forward looking health-related prediction in accordance with aspects of the present disclosure. The device 805 may be an example of or include the components of a device 505 as described herein. The device 805 may include an example of a wearable device 104, as described previously herein. The device 805 may include components for bi-directional communications including components for transmitting and receiving communications with a user device 106 and a server 110, such as a wearable device manager 820, a communication module 810, an antenna 815, a sensor component 825, a power module 830, a memory 835, a processor 840, and a wireless device 850. These components may be in electronic communication or otherwise coupled (e.g., operatively, communicatively, functionally, electronically, electrically) via one or more buses (e.g., a bus 845).

The wearable device manager 820 may support generating personalized health-related predictions from measured physiological data in accordance with examples as disclosed herein. For example, the wearable device manager 820 may be configured as or otherwise support a means for receiving, from a wearable device, first physiological data measured from a user via the wearable device throughout a first time interval. The wearable device manager 820 may be configured as or otherwise support a means for outputting, via a machine learning model based on inputting the first physiological data into the machine learning model, one or more health-related predictions associated with the user during a second time interval, wherein the one or more health-related predictions comprise a predicted change in a health-related metric during the second time interval from a first predicted value to a second predicted value based at least in part on one or more hypothetical user actions engaged in by the user between the first time interval and the second time interval. The wearable device manager 820 may be configured as or otherwise support a means for causing a GUI of a user device associated with the wearable device to display information associated with the one or more health-related predictions prior to the second time interval.

By including or configuring the wearable device manager 820 in accordance with examples as described herein, the device 805 may support techniques for generating forward-looking health-related predictions which may improve user experience related to understanding how their choices (e.g., actions) impact their overall health, increase user motivation to proactively make choices to improve their overall health, and increase user engagement with tags, among other advantages.

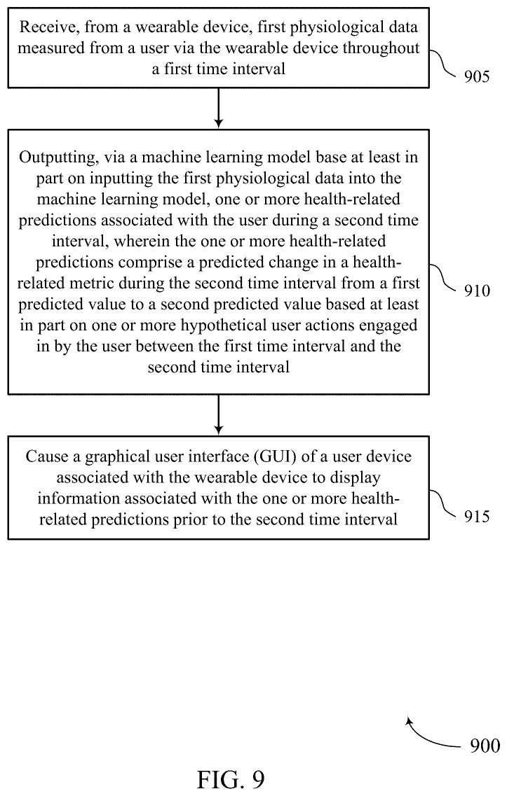

FIG. 9 shows a flowchart illustrating a method 900 that supports forward looking health-related prediction in accordance with aspects of the present disclosure. The operations of the method 900 may be implemented by a user device or a wearable device or its components as described herein. For example, the operations of the method 900 may be performed by a user device or a wearable device as described with reference to FIGS. 1 through 8. In some examples, a user device or a wearable device may execute a set of instructions to control the functional elements of the wireless user device or the wireless wearable device to perform the described functions. Additionally, or alternatively, the wireless user device or the wireless wearable device may perform aspects of the described functions using special-purpose hardware.

At 905, the method may include receiving, from a wearable device, first physiological data measured from a user via the wearable device throughout a first time interval. The operations of 905 may be performed in accordance with examples as disclosed herein. In some examples, aspects of the operations of 905 may be performed by a data acquisition manager 625 as described with reference to FIG. 6.

At 910, the method may include outputting, via a machine learning model based at least in part on inputting the first physiological data into the machine learning model, one or more health-related predictions associated with the user during a second time interval, wherein the one or more health-related predictions comprise a predicted change in a health-related metric during the second time interval from a first predicted value to a second predicted value based at least in part on one or more hypothetical user actions engaged in by the user between the first time interval and the second time interval. The operations of 910 may be performed in accordance with examples as disclosed herein. In some examples, aspects of the operations of 910 may be performed by a prediction manager 630 as described with reference to FIG. 6.

At 915, the method may include causing a GUI of a user device associated with the wearable device to display information associated with the one or more health-related predictions prior to the second time interval. The operations of 915 may be performed in accordance with examples as disclosed herein. In some examples, aspects of the operations of 915 may be performed by a user interface manager 635 as described with reference to FIG. 6.

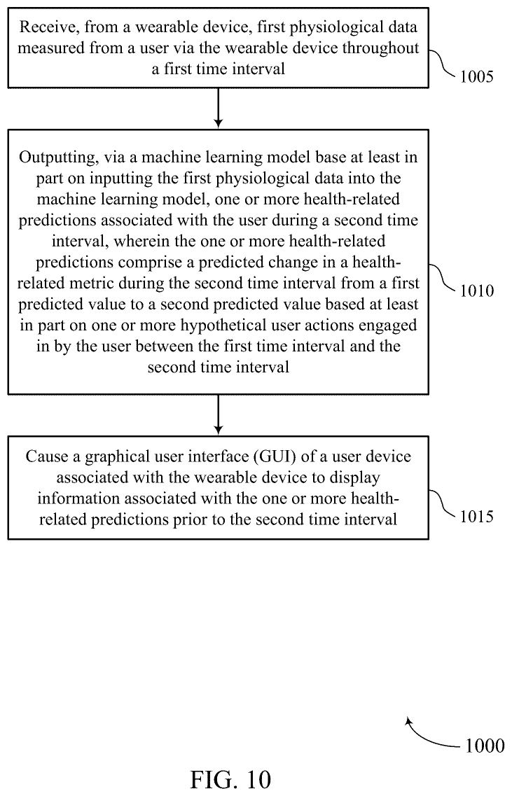

FIG. 10 shows a flowchart illustrating a method 1000 that supports forward looking health-related prediction in accordance with aspects of the present disclosure. The operations of the method 1000 may be implemented by a user device or a wearable device or its components as described herein. For example, the operations of the method 1000 may be performed by a user device or a wearable device as described with reference to FIGS. 1 through 8. In some examples, a user device or a wearable device may execute a set of instructions to control the functional elements of the wireless user device or the wireless wearable device to perform the described functions. Additionally, or alternatively, the wireless user device or the wireless wearable device may perform aspects of the described functions using special-purpose hardware.

At 1005, the method may include receiving, from a wearable device, first physiological data measured from a user via the wearable device throughout a first time interval. The operations of 1005 may be performed in accordance with examples as disclosed herein. In some examples, aspects of the operations of 1005 may be performed by a data acquisition manager 625 as described with reference to FIG. 6.

At 1010, the method may include outputting, via a machine learning model based at least in part on inputting the first physiological data into the machine learning model, one or more health-related predictions associated with the user during a second time interval, wherein the one or more health-related predictions comprise a predicted change in a health-related metric during the second time interval from a first predicted value to a second predicted value based at least in part on one or more hypothetical user actions engaged in by the user between the first time interval and the second time interval. The operations of 1010 may be performed in accordance with examples as disclosed herein. In some examples, aspects of the operations of 1010 may be performed by a prediction manager 630 as described with reference to FIG. 6.

At 1015, the method may include causing a GUI of a user device associated with the wearable device to display information associated with the one or more health-related predictions prior to the second time interval. The operations of 1015 may be performed in accordance with examples as disclosed herein. In some examples, aspects of the operations of 1015 may be performed by a user interface manager 635 as described with reference to FIG. 6.

It should be noted that the methods described above describe possible implementations, and that the operations and the steps may be rearranged or otherwise modified and that other implementations are possible. Furthermore, aspects from two or more of the methods may be combined.

A method for generating personalized health-related predictions from measured physiological data is described. The method may include receiving, from a wearable device, first physiological data measured from a user via the wearable device throughout a first time interval, outputting, via a machine learning model based at least in part on inputting the first physiological data into the machine learning model, one or more health-related predictions associated with the user during a second time interval, wherein the one or more health-related predictions comprise a predicted change in a health-related metric during the second time interval from a first predicted value to a second predicted value based at least in part on one or more hypothetical user actions engaged in by the user between the first time interval and the second time interval, and causing a GUI of a user device associated with the wearable device to display information associated with the one or more health-related predictions prior to the second time interval.

An apparatus for generating personalized health-related predictions from measured physiological data is described. The apparatus may include a processor, memory coupled with the processor, and instructions stored in the memory. The instructions may be executable by the processor to cause the apparatus to receive, from a wearable device, first physiological data measured from a user via the wearable device throughout a first time interval, outputting, via a machine learning model base at least in part on inputting the first physiological data into the machine learning model, one or more health-related predictions associated with the user during a second time interval, wherein the one or more health-related predictions comprise a predicted change in a health-related metric during the second time interval from a first predicted value to a second predicted value based at least in part on one or more hypothetical user actions engaged in by the user between the first time interval and the second time interval, and cause a GUI of a user device associated with the wearable device to display information associated with the one or more health-related predictions prior to the second time interval.

Another apparatus for generating personalized health-related predictions from measured physiological data is described. The apparatus may include means for receiving, from a wearable device, first physiological data measured from a user via the wearable device throughout a first time interval, means for outputting, via a machine learning model based at least in part on inputting the first physiological data into the machine learning model, one or more health-related predictions associated with the user during a second time interval, wherein the one or more health-related predictions comprise a predicted change in a health-related metric during the second time interval from a first predicted value to a second predicted value based at least in part on one or more hypothetical user actions engaged in by the user between the first time interval and the second time interval, and means for causing a GUI of a user device associated with the wearable device to display information associated with the one or more health-related predictions prior to the second time interval.

A non-transitory computer-readable medium storing code for generating personalized health-related predictions from measured physiological data is described. The code may include instructions executable by a processor to receive, from a wearable device, first physiological data measured from a user via the wearable device throughout a first time interval, outputting, via a machine learning model base at least in part on inputting the first physiological data into the machine learning model, one or more health-related predictions associated with the user during a second time interval, wherein the one or more health-related predictions comprise a predicted change in a health-related metric during the second time interval from a first predicted value to a second predicted value based at least in part on one or more hypothetical user actions engaged in by the user between the first time interval and the second time interval, and cause a GUI of a user device associated with the wearable device to display information associated with the one or more health-related predictions prior to the second time interval.

Some examples of the method, apparatuses, and non-transitory computer-readable medium described herein may further include operations, features, means, or instructions for receiving, via the user device, a user input indicating one or more additional hypothetical user actions to be performed by the user subsequent to the first time interval and prior to the second time interval, outputting, from the machine learning model based at least in part on inputting the user input to the machine learning model, one or more modifications to the one or more health-related predictions, and causing the GUI of the user device to display additional information associated with the one or more modifications to the one or more health-related predictions.

In some examples of the method, apparatuses, and non-transitory computer-readable medium described herein, the user input further indicates timing information associated with the one or more additional hypothetical user actions.

In some examples of the method, apparatuses, and non-transitory computer-readable medium described herein, the one or more additional hypothetical user actions comprise a hypothetical bedtime, a hypothetical wake time, a hypothetical workout, a hypothetical nap, a hypothetical meal consumption, a hypothetical caffeine consumption, a hypothetical alcohol consumption, or any combination thereof.

Some examples of the method, apparatuses, and non-transitory computer-readable medium described herein may further include operations, features, means, or instructions for receiving, from the wearable device, baseline physiological data measured from the user via the wearable device prior to the first time interval and determining one or more characteristics of a routine of the user based at least in part on the baseline physiological data, wherein the one or more hypothetical user actions may be based at least in part on the one or more characteristics of the routine of the user.

In some examples of the method, apparatuses, and non-transitory computer-readable medium described herein, the one or more characteristics of the routine comprise a bedtime, a wake-time, a workout timing, a meditation timing, a workout type, a meal timing, a nap timing, a nap duration, or any combination thereof.

Some examples of the method, apparatuses, and non-transitory computer-readable medium described herein may further include operations, features, means, or instructions for causing the GUI of the user device to display the one or more hypothetical user actions used to generate the one or more health-related predictions.

Some examples of the method, apparatuses, and non-transitory computer-readable medium described herein may further include operations, features, means, or instructions for receiving, from the wearable device, second physiological data measured from the user via the wearable device subsequent to the first time interval and prior to the second time interval, outputting, from the machine learning model based at least in part on inputting the second physiological data to the machine learning model, one or more modifications to the one or more health-related predictions, and causing the GUI of the user device to display additional information associated with the one or more modifications to the one or more health-related predictions.

Some examples of the method, apparatuses, and non-transitory computer-readable medium described herein may further include operations, features, means, or instructions for receiving, via the user device, a user input indicating one or more tags associated with the user subsequent to the first time interval and prior to the second time interval, outputting, from the machine learning model based at least in part on inputting the one or more tags to the machine learning model, one or more modifications to the one or more health-related predictions, and causing the GUI of the user device to display additional information associated with the one or more modifications to the one or more health-related predictions.

Some examples of the method, apparatuses, and non-transitory computer-readable medium described herein may further include operations, features, means, or instructions for determining one or more recommended actions to preempt, adjust, or maintain the predicted change in the health-related metric prior to the second time interval and causing the GUI of the user device associated with the wearable device to display the one or more recommended actions to preempt, adjust, or maintain the predicted change in the health-related metric prior to the second time interval.

Some examples of the method, apparatuses, and non-transitory computer-readable medium described herein may further include operations, features, means, or instructions for receiving sensor data from the wearable device, the user device, or both, the sensor data associated with one or more characteristics of a physical environment of the user during the first time interval, wherein the one or more recommended actions comprise a recommended modification to the one or more characteristics of the physical environment of the user during the second time interval.

Some examples of the method, apparatuses, and non-transitory computer-readable medium described herein may further include operations, features, means, or instructions for receiving, via the user device, a user input indicating a desired value of health-related metric during the second time interval, determining one or more recommended actions to achieve the desired value of the health-related metric during the second time interval, wherein the one or more recommended actions may be based at least in part on a difference between the second predicted value and the desired value, and causing the GUI of the user device associated with the wearable device to display the one or more recommended actions to achieve the desired value of the health-related metric during the second time interval.

Some examples of the method, apparatuses, and non-transitory computer-readable medium described herein may further include operations, features, means, or instructions for receiving second physiological data measured from the user via the wearable device throughout the time interval, calculating an actual value of the health-related metric during the second time interval based at least in part on the second physiological data, and adjusting one or more parameters of the machine learning model based at least in part on a comparison between the actual value of the health-related metric during the second time interval and the second predicted value of the health-related metric during the second time interval.

Some examples of the method, apparatuses, and non-transitory computer-readable medium described herein may further include operations, features, means, or instructions for receiving, prior to the second time interval, user data associated with the user from one or more applications associated with the wearable device, the user device, or both, the one or more applications comprising a lifestyle application, a social media application, a utility application, an entertainment application, a productivity application, an information outlet application, or any combination thereof and inputting the user data into the machine learning model, wherein the one or more health-related predictions associated with the user during the second time interval may be based at least in part on inputting the user data to the machine learning model.

In some examples of the method, apparatuses, and non-transitory computer-readable medium described herein, the predicted change in the health-related metric during the second time interval may be based at least in part on timing information associated with the one or more hypothetical user actions engaged in by the user between the first time interval and the second time interval.

In some examples of the method, apparatuses, and non-transitory computer-readable medium described herein, the machine learning model may be trained based at least in part on data associated with the user, data associated with a set of users, or both.

In some examples of the method, apparatuses, and non-transitory computer-readable medium described herein, the first physiological data measured from the user via the wearable device throughout the first time interval comprises data associated with a Sleep Score of the user, a Readiness Score of the user, or both.

In some examples of the method, apparatuses, and non-transitory computer-readable medium described herein, the data associated with a Sleep Score of the user comprises data associated with a circadian rhythm of the user.

In some examples of the method, apparatuses, and non-transitory computer-readable medium described herein, the wearable device comprises a ring wearable device.

The description set forth herein, in connection with the appended drawings, describes example configurations and does not represent all the examples that may be implemented or that are within the scope of the claims. The term “exemplary” used herein means “serving as an example, instance, or illustration,” and not “preferred” or “advantageous over other examples.” The detailed description includes specific details for the purpose of providing an understanding of the described techniques. These techniques, however, may be practiced without these specific details. In some instances, well-known structures and devices are shown in block diagram form in order to avoid obscuring the concepts of the described examples.

In the appended figures, similar components or features may have the same reference label. Further, various components of the same type may be distinguished by following the reference label by a dash and a second label that distinguishes among the similar components. If just the first reference label is used in the specification, the description is applicable to any one of the similar components having the same first reference label irrespective of the second reference label.

Information and signals described herein may be represented using any of a variety of different technologies and techniques. For example, data, instructions, commands, information, signals, bits, symbols, and chips that may be referenced throughout the above description may be represented by voltages, currents, electromagnetic waves, magnetic fields or particles, optical fields or particles, or any combination thereof.

The various illustrative blocks and modules described in connection with the disclosure herein may be implemented or performed with a general-purpose processor, a DSP, an ASIC, an FPGA or other programmable logic device, discrete gate or transistor logic, discrete hardware components, or any combination thereof designed to perform the functions described herein. A general-purpose processor may be a microprocessor, but in the alternative, the processor may be any conventional processor, controller, microcontroller, or state machine. A processor may also be implemented as a combination of computing devices (e.g., a combination of a DSP and a microprocessor, multiple microprocessors, one or more microprocessors in conjunction with a DSP core, or any other such configuration).

The functions described herein may be implemented in hardware, software executed by a processor, firmware, or any combination thereof. If implemented in software executed by a processor, the functions may be stored on or transmitted over as one or more instructions or code on a computer-readable medium. Other examples and implementations are within the scope of the disclosure and appended claims. For example, due to the nature of software, functions described above can be implemented using software executed by a processor, hardware, firmware, hardwiring, or combinations of any of these. Features implementing functions may also be physically located at various positions, including being distributed such that portions of functions are implemented at different physical locations. Also, as used herein, including in the claims, “or” as used in a list of items (for example, a list of items prefaced by a phrase such as “at least one of” or “one or more of”) indicates an inclusive list such that, for example, a list of at least one of A, B, or C means A or B or C or AB or AC or BC or ABC (i.e., A and B and C). Also, as used herein, the phrase “based on” shall not be construed as a reference to a closed set of conditions. For example, an exemplary step that is described as “based on condition A” may be based on both a condition A and a condition B without departing from the scope of the present disclosure. In other words, as used herein, the phrase “based on” shall be construed in the same manner as the phrase “based at least in part on.”

Computer-readable media includes both non-transitory computer storage media and communication media including any medium that facilitates transfer of a computer program from one place to another. A non-transitory storage medium may be any available medium that can be accessed by a general purpose or special purpose computer. By way of example, and not limitation, non-transitory computer-readable media can comprise RAM, ROM, electrically erasable programmable ROM (EEPROM), compact disk (CD) ROM or other optical disk storage, magnetic disk storage or other magnetic storage devices, or any other non-transitory medium that can be used to carry or store desired program code means in the form of instructions or data structures and that can be accessed by a general-purpose or special-purpose computer, or a general-purpose or special-purpose processor. Also, any connection is properly termed a computer-readable medium. For example, if the software is transmitted from a website, server, or other remote source using a coaxial cable, fiber optic cable, twisted pair, digital subscriber line (DSL), or wireless technologies such as infrared, radio, and microwave, then the coaxial cable, fiber optic cable, twisted pair, DSL, or wireless technologies such as infrared, radio, and microwave are included in the definition of medium. Disk and disc, as used herein, include CD, laser disc, optical disc, digital versatile disc (DVD), floppy disk and Blu-ray disc where disks usually reproduce data magnetically, while discs reproduce data optically with lasers. Combinations of the above are also included within the scope of computer-readable media.

The description herein is provided to enable a person skilled in the art to make or use the disclosure. Various modifications to the disclosure will be readily apparent to those skilled in the art, and the generic principles defined herein may be applied to other variations without departing from the scope of the disclosure. Thus, the disclosure is not limited to the examples and designs described herein, but is to be accorded the broadest scope consistent with the principles and novel features disclosed herein.

What is claimed is:

1. A method for generating personalized health-related predictions from measured physiological data, comprising:
receiving, from a wearable device, first physiological data measured from a user via the wearable device throughout a first time interval;
outputting, via a machine learning model based at least in part on inputting the first physiological data into the machine learning model, one or more health-related predictions associated with the user during a second time interval, wherein the one or more health-related predictions comprise a predicted change in a health-related metric during the second time interval from a first predicted value to a second predicted value based at least in part on one or more expected user actions engaged in by the user between the first time interval and the second time interval; and
causing a user interface of a user device associated with the wearable device to display, prior to the second time interval, information associated with the one or more health-related predictions.

2. The method of claim 1, further comprising:
receiving, via the user device, a user input indicating one or more additional expected user actions to be performed by the user subsequent to the first time interval and prior to the second time interval;
outputting, from the machine learning model based at least in part on inputting the user input to the machine learning model, one or more modifications to the one or more health-related predictions; and
causing the user interface of the user device to display additional information associated with the one or more modifications to the one or more health-related predictions.

3. The method of claim 2, wherein the user input further indicates timing information associated with the one or more additional expected user actions.

4. The method of claim 2, wherein the one or more additional expected user actions comprise an expected bedtime, an expected wake time, an expected workout, an expected nap, an expected meal consumption, an expected caffeine consumption, an expected alcohol consumption, or any combination thereof.

5. The method of claim 1, further comprising:
receiving, from the wearable device, baseline physiological data measured from the user via the wearable device prior to the first time interval; and
determining one or more characteristics of a routine of the user based at least in part on the baseline physiological data, wherein the one or more expected user actions are based at least in part on the one or more characteristics of the routine of the user.

6. The method of claim 5, wherein the one or more characteristics of the routine comprise a bedtime, a wake-time, a workout timing, a meditation timing, a workout type, a meal timing, a nap timing, a nap duration, or any combination thereof.

7. The method of claim 1, further comprising:
causing the user interface of the user device to display the one or more expected user actions used to generate the one or more health-related predictions.

8. The method of claim 1, further comprising:
receiving, from the wearable device, second physiological data measured from the user via the wearable device subsequent to the first time interval and prior to the second time interval;
outputting, from the machine learning model based at least in part on inputting the second physiological data to the machine learning model, one or more modifications to the one or more health-related predictions; and
causing the user interface of the user device to display additional information associated with the one or more modifications to the one or more health-related predictions.

9. The method of claim 1, further comprising:
receiving, via the user device, a user input indicating one or more tags associated with the user subsequent to the first time interval and prior to the second time interval;
outputting, from the machine learning model based at least in part on inputting the one or more tags to the machine learning model, one or more modifications to the one or more health-related predictions; and
causing the user interface of the user device to display additional information associated with the one or more modifications to the one or more health-related predictions.

10. The method of claim 1, further comprising:
determining one or more recommended actions to preempt, adjust, or maintain the predicted change in the health-related metric prior to the second time interval; and
causing the user interface of the user device associated with the wearable device to display the one or more recommended actions to preempt, adjust, or maintain the predicted change in the health-related metric prior to the second time interval.

11. The method of claim 10, further comprising:
receiving sensor data from the wearable device, the user device, or both, the sensor data associated with one or more characteristics of a physical environment of the user during the first time interval, wherein the one or more recommended actions comprise a recommended modification to the one or more characteristics of the physical environment of the user during the second time interval.

12. The method of claim 1, further comprising:
receiving, via the user device, a user input indicating a desired value of health-related metric during the second time interval;
determining one or more recommended actions to achieve the desired value of the health-related metric during the second time interval, wherein the one or more recommended actions are based at least in part on a difference between the second predicted value and the desired value; and
causing the user interface of the user device associated with the wearable device to display the one or more recommended actions to achieve the desired value of the health-related metric during the second time interval.

13. The method of claim 1, further comprising:
receiving second physiological data measured from the user via the wearable device throughout the time interval;
calculating an actual value of the health-related metric during the second time interval based at least in part on the second physiological data; and
adjusting one or more parameters of the machine learning model based at least in part on a comparison between the actual value of the health-related metric during the second time interval and the second predicted value of the health-related metric during the second time interval.

14. The method of claim 1, further comprising:
receiving, prior to the second time interval, user data associated with the user from one or more applications associated with the wearable device, the user device, or both, the one or more applications comprising a lifestyle application, a social media application, a utility application, an entertainment application, a productivity application, an information outlet application, or any combination thereof; and
inputting the user data into the machine learning model, wherein the one or more health-related predictions associated with the user during the second time interval are based at least in part on inputting the user data to the machine learning model.

15. The method of claim 1, wherein the predicted change in the health-related metric during the second time interval is based at least in part on timing information associated with the one or more expected user actions engaged in by the user between the first time interval and the second time interval.

16. The method of claim 1, wherein the machine learning model is trained based at least in part on data associated with the user, data associated with a set of users, or both.

17. The method of claim 1, wherein the first physiological data measured from the user via the wearable device throughout the first time interval comprises data associated with a Sleep Score of the user, a Readiness Score of the user, or both.

18. The method of claim 17, wherein the data associated with a Sleep Score of the user comprises data associated with a circadian rhythm of the user.

19. The method of claim 1, wherein the wearable device comprises a ring wearable device.
